(19) United States
(12) Patent Application Publication
(10) Pub. No.: US 2003/0166599 A1
(43) Pub. Date: Sep. 4, 2003

(54) Uses of tgap7 for the modulation of leucocyte activation

(76) Inventors: Nalan Utku, Berlin (DE); Mirko Schlawinsky, Berlin (DE)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/275,140

(22) PCT Filed: May 3, 2001
(86) PCT No.: PCT/EP01/05010

(30) Foreign Application Priority Data
May 3, 2000 (EP) 00109462.2

(51) Int. Cl. A61K 48/00

(52) U.S. Cl. 514/44

(57) ABSTRACT

Described are pharmaceutical compositions comprising polynucleotides encoding TGAP7 protein or a biologically fragment thereof. Furthermore, pharmaceutical compositions are described which comprise vectors comprising the aforementioned polynucleotides and/or host cells transformed therewith. Additionally, pharmaceutical compositions comprising antisense constructs to be aforementioned polynucleotides are described. Furthermore, methods and uses for modulating immune responses through the TGAP7 protein as well as pharmaceutical compositions comprising agents which act on the TGAP7 protein are described. Also, the use of said polynucleotide, vector, protein or antibody for the preparation of diagnostic and pharmaceutical compositions for use, inter alia, in organ transplantation, for the treatment of autoimmune, allergic or infectious diseases, or for treatment of tumors is provided.

### FIELD OF THE INVENTION

The present invention relates to pharmaceutical compositions comprising polynucleotides encoding a TGAP7 protein or a biologically active fragment thereof. Furthermore, the present invention relates to pharmaceutical compositions comprising a nucleic acid molecule of at least 15 nucleotides in length hybridizing specifically with a polynucleotide described herein or with a complementary strand thereof. In addition, the present invention pertains to pharmaceutical compositions comprising vectors containing polynucleotides encoding a TGAP7 protein, comprising (host-)cells which comprise said polynucleotide(s) or said vectors, comprising a TGAP7 protein or biologically active fragments thereof, comprising an antibody which specifically recognizes a TGAP7 protein or a fragment thereof, or comprising an antisense construct capable of inhibiting the expression of a polynucleotide encoding a TGAP7 protein. Additonally, this invention provides diagnostic compositions and methods of diagnosing biologically conditions. Also, the invention relates to methods for identifying binding partners to a TGAP7 protein and to methods for identifying leucocyte activating or co-stimulating compounds or for identifying inhibitors of leucocyte activation and stimulation. Finally, the present invention relates to the use of the before described polynucleotide(s), vector(s), protein(s), antisense construct(s) for the preparation of compositions for diagnosing or the treatment of acute and chronic diseases, involving T-cell activation and Th1 and Th2 immune response, for the treatment of acute and chronic rejection of allo- and xeno-organ transplants and bone marrow transplantation, for the treatment of rheumatoid arthritis, lupus erythramatodes, multiple sklerosis, encephalitis, vasculitis, diabetes mellitus, pancreatitis, gastritis, thyroiditis, for the treatment of (maligne) disorders of T, B or NK cells, for the treatment of asthma, lepramatosis, Helicobacter pylori associated gastritis or for the treatment of skin tumors, adrenal tumors or lung tumors, wound healing, growth disorders, inflammatory and/or infectious diseases.

Several documents are cited throughout the text of this specification. Each of the documents cited herein (including any manufacturer's specifications, instructions, etc.) are hereby incorporated herein by reference; however, there is no admission that any document cited is indeed prior art as to the present invention.

### BACKGROUND OF THE INVENTION

T-cell activation is accompanied with sequential changes in the expression of various genes over several days and involves multiple signaling pathways [1]. Stimulation of T-cells is initiated by the interaction of antigen-specific T-cell receptors (TCR) with MHC bound antigenic peptides presented on the surface of antigen presenting cells (APC), but full proliferative T-cell response requires additional costimulatory signals which are provided by the interaction of proteins expressed on the surface of T-cells and APC [2,3,4,5]. In addition, a number of cytokines as well as other proteins are known to augment T-cell activation, although many of them appear not to be essential for the basic proliferative T-cell response [3,6]. Moreover, a growing body of evidence indicates that the microtubule cytoskeleton of lymphocytes plays a major role in T-cell activation. Stimulation of T-cells was demonstrated to result in molecular rearrangement in the actin cytoskeleton leading to re-localization and concentration of signaling molecules in restricted areas of the cell membrane close to the bound APC [7,8,9].

Although considerable information on T-cell activation has been gathered in recent years, the complex molecular mechanisms of stimulation and signaling pathways are not completely understood. Since T-cell activation provides the central event in various types of inflammation as well as in autoimmune disease and graft rejection, knowledge about the distinct steps and molecules involved in the stimulation process is of considerable biomedical importance, as they might provide targets for therapeutic modulation of the immune response. Therapeutic prevention of T-cell activation in organ transplantation and autoimmune diseases presently relies on panimmunosuppressive drugs interfering with downstream intracellullar events.

Alloreactive CD4 or CD8 cells or specific alloantibodies are capable of mediating, inter alia, allograft rejection. The following immune mechanisms cause graft rejection by different mechanisms:

(a) alloactive T-cells can recruit and activate macrophages, initiating graft injury by “delayed-type” hypersensitivity response;

(b) alloactive cytotoxic T-cells are capable of directly lysing graft endothelial and parenchymal cells; and

(c) alloantibodies bind to endothelium, activate the complement system, and injure thereby graft blood vessels.

The various forms of (allograft) rejection imply a temporal sequence of events including hyperacute, acute vascular and acute cellular as well as chronic rejection.

Hyperacute rejection plays an important role in xenotransplantation and is due to natural antibodies (Ref: Milford, E., Utku A, N. Guidelines for use of immunogenetic tests in organ transplanation, Manual of Clinical Laboratory Immunology, ASM Press 1997). Hyperacute rejection is mediated by preexisting antibodies that bind to endothelium and activate complement and is characterized by rapid thrombotic occlusion of the graft vasculature. In more recent clinical experience, hyperacute rejection of allografts is usually mediated by antibodies directed against protein alloantigens, such as foreign MHC molecules, or against less well described alloantigens expressed on vascular endothelial cells. Such antibodies generally arise as a result of prior exposure to alloantigens through blood transfusion, prior transplantation, or multiple pregnancies (loc. cit.). These antibodies are often of the IgG type. By testing recipients for the presence of such reactive antibodies with the cells of potential donors, hyperacute rejection has been virtually eliminated from clinical allo-transplantation but remains a major problem in xenotransplantation.

Acute vascular rejection is mediated by IgG antibodies produced by B-cells against endothelial alloantigens and involves activation of complement. T-cells contribute to vascular injury by responding to alloantigens present on endothelial cells, leading to direct cell lysis of these cells, or the production of cytokines that recruit and activate inflammatory cells.

Acute cellular rejection is characterized by necrosis of parenchymal cells and is usually accompanied by lymphocyte and macrophage infiltrates. These infiltrating leucocytes are responsible for the lysis of the graft. Several different effector mechanisms may be involved in acute cellular rejection including CTL-mediated lysis, activated-macrophage-mediated lysis (as delayed type hypersensibility, DTH), and natural killer cells mediated lysis.

The identification of both antibody and lymphocytes as important effector mechanism in acute graft rejection suggests that this process is similar to normal antiviral immune responses. The basis of similarity probably arises from the fact that the foreign class I MHC molecules present in the graft are recognized as if they were self MHC molecules associated with endogenously synthesized foreign peptides.

Chronic rejection is characterized by fibrosis with loss of normal organ structures. The fibrosis may represent wound healing following the cellular necrosis of acute rejection or a form of DTH in which activated macrophages secrete mesenchymal cell growth factors, or alternatively chronic rejection is a response to chronic ischemia caused by injury of blood vessels. Vascular occlusion is due to proliferation of intimal smooth muscle cells, called accelerated or graft arteriosclerosis. This features of chronic inflammation is characterized by fibrosis in autoimmune diseases such as lupus erythramatodes, sklerodermia and panarteriitis nodosa. Chronic inflammatory immune response involves all parts of cellular and humoral immune system (T-, B-, NK- cells, monocytes) and is the response to the extensive production of autoantibodies and creation of immune complexes against different cellular components. This leads to multiple organ failure caused by significant tissues damage such as myositis, polyneuropathia, heart disease, vasculitis, etc.

Considering, inter alia, the above described temporal sequence events in allograft and xenograft rejection, it is desired to specifically modulate lymphocyte cell responses, i.e. to modulate T-, B-, NK-cells and/or monocyte responses during immunological processes. Furthermore, it is desired to specifically modulate immunological processes like, inter alia, autoimmunological events.

Specific modulation of the immune response remains, therefore, a longstanding goal in immunological research.

### SUMMARY OF THE INVENTION

The present invention relates to pharmaceutical compositions comprising polynucleotides encoding an immune response modulating protein TGAP7. Furthermore, the present invention relates to pharmaceutical compositions comprising peptides and polypeptides derived therefrom as well as to pharmaceutical compositions comprising antibodies capable of inhibiting leucocyte stimulation through the immune response modulating protein TGAP7. More particularly, the present invention relates to applications in the medical field that directly arise from the polynucleotides, protein, peptides, (poly)peptides, antisense constructs and antibodies described in this invention. Additionally, the present invention relates to a novel method for testing activators and inhibitors of leucocyte proliferation, i.e. of leucocyte activation and/or stimulation. The pharmaceutical compositions, methods and uses of the invention are useful therapeutically and/or diagnostically in situations where it is desirable to modulate (antigen-specific) immune responses, e.g., inducing and maintain (antigen-specific) T-cell or B-cell non/unresponsiveness, wherein said non/unresponsiveness comprises the selective inhibition of immune cell subsets which are able of creating a response to specific antigen(s), inter alia, antigen(s) in transplanted tissue. The pharmaceutical compositions, methods and uses of the invention are furthermore useful to restore (antigen-specific) B or T-cell responsiveness. For example, it may be necessary to induce or maintain “selective immune” unresponsiveness in a subject who has received an organ or bone marrow transplant to prevent graft rejection by inhibiting stimulation through the TGAP7 protein in cells of the immune system such as T-cells, B-cells, NK-cells, monocytes and/or macrophages. In addition, T-cell unresponsiveness can be maintained by blocking TGAP7 stimulation in a subject who has an autoimmune disease to alleviate symptoms of the autoimmune disease. In these cases, a TGAP7 inhibitory agent is administered to the subject in an amount and over a period of time sufficient to maintain T-cell unresponsiveness. Alternatively, T-cell unresponsiveness can be reversed in a subject bearing a tumor to stimulate a tumor specific NK- and T-cell response or in a subject receiving a vaccine to enhance the efficacy of the vaccine. For example, it might be useful to induce or maintain the status of activation of the immune cells through vaccination with TGAP7 peptides in a subject who developed a tumor to orchestrate the enhancement of immune response in T-, B-, NK-cells and/or monocytes.

### DETAILED DESCRIPTION OF THE PRESENT INVENTION

In view of the need of therapeutic means for the diagnosis and treatment of diseases related to immune responses of the human body, the technical problem of the invention is to provide means and methods for the modulation of immune cell responses which are particularly useful in organ transplantation and autoimmune diseases.

The solution to this technical problem is achieved by providing the embodiments characterized in the claims, namely novel pharmaceutical compositions comprising an immune response modulating protein encoded by a immune response cDNA designated “TGAP7” which exhibits a central role in leucocyte activation and growth, wherein said leucocyte activation refers to the activation of T-, B-, NK-cells and/or monocytes. TGAP7 mRNA is transiently upregulated in the early phase of T-cell activation.

In a first set of experiments, the TGAP7 protein encoding cDNA has been cloned and characterized; see Example 1. Furthermore, the expression pattern of TGAP7 was investigated after allo-stimulation of human leucocytes at time points 0, 1, 24 and 72 h and results obtained with alloactivated T-cells revealed an upregulation of TGAP7 only after 24 h after immune activation of the TGAP7 gene. It is thus an excellent marker for diagnosis of the status of immune response in a subject.

An independent set of experiments performed in accordance with the present invention, surprisingly revealed that modulation of the TGAP7 expression with specific TGAP7 antisense polynucleotides efficiently leads to a significant down regulation of lymphocyte activation in response to allo- and mitogens. The latter described results obtained in accordance with the present invention provide evidence for an essential role of TGAP7 in the early events of leucocyte activation. Thus, targeting of TGAP7 protein and its encoding gene provides a novel therapeutic approach for modulation of the immune response. Accordingly, the invention relates to a pharmaceutical composition comprising a polynucleotide encoding a TGAP7 protein or a biologically active fragment thereof comprising a nucleic acid sequence selected from the group consisting of:

(i) DNA sequences comprising a nucleotide sequence encoding the amino acid sequence depicted in SEQ ID NO: 2 or 4;

(ii) DNA sequences comprising the nucleotide sequence depicted in SEQ ID NO: 1 or 3;

(iii) DNA sequences comprising a nucleotide sequence encoding a fragment or derivative of the protein encoded by the DNA sequence of (i) or (ii);

(iv) DNA sequences the complementary strand of which hybridizes with and which is at least 70% identical to the polynucleotide as defined in any one of (i) to (iii); and

(v) DNA sequences the nucleotide of which is degenerate to the nucleotide sequence of a DNA sequence of any one of (i) to (iv);

The term “TGAP7 protein”, in accordance with the present invention, denotes a protein involved in the signal transduction of leucocyte activation and/or proliferation and down-regulation of which results in suppressing leucocyte, preferably T-, B-, NK-cell and/or monocyte proliferation in response to alloactivation in a mixed lymphocyte culture or in response to mitogens when exogeneously added to the culture. In accordance with this invention it has been surprisingly found that a cDNA is differentially expressed in alloactivated leucocytes, i.e. human T-cells. This differentially expressed cDNA was termed TGAP7 and it was shown that this cDNA encodes a protein sequence which is 100% identical to a putative GAP protein E6TPI (see Gao (1999), MCB 19, 733-744). In accordance with the present inventon, it has surprisingly been found that said protein/polypeptide plays an important role in the differentiation of quiescent T-cells to activate T-cells after allo-/autoantigen stimulation and/or cell activation/proliferation processes of B-cells, NK-cells and/or monocytes after stimulation by said allo-/autoantigen or by antigens from, inter alia, pathological agents, like viruses (viral agents), bacteria, etc. The term “TGAP7” denotes proteins/polypeptides, in accordance with this invention, which are identical to the TGAP7-α or TGAP7-β proteins/polypeptides as described herein (see SEQ ID NOs: 2, 4, 5 or 6 and FIG. 1) and the term comprises, furthermore, functional homologues of said protein/polypeptide.

Studies which had been carried out within the scope of the present invention revealed that antisense polynucleotides directed to the mRNA encoding TGAP7 protein are able to efficiently suppress the proliferation of lymphocytes and antigen presenting cells (monocytes, dentritic cells, B-cells) in response to alloactivation in a mixed lymphocyte culture or in response to mitogens. Alloantigen and mitogen (ConA and PHA) stimulated human lymphocytes and monocytes were incubated in the presence and absence of TGAP7 specific antisense oligonucleotides for 24, 48, 72, 96 and 168 h. For proliferation, thymidine uptake was determined after 6 h which demonstrated a significant inhibition of T-cell activation in the presence TGAP7-specific antisense oligonucleotides whereas the presence of unrelated control antisense oligonucleotides did not exhibit any effect on T-cell proliferation. For activation studies early T-cell specific markers such as CD69 and IL2 receptor were analyzed by flowcytometry on human peripheral blood lymphocytes in the presence and absence of TGAP7 specific antisense oligonucleotides which demonstrate significant downregulation of activation markers (like e.g. CD25-(IL2 receptor), CD69, L/CA class II and transferrin receptor) on immune cells. Based on these results, it can be concluded that TGAP7 molecule is directly involved in the initiating of the immune response and might be an important target molecule for modulating the immune response.

The term “leucocytes” generally denotes all kinds of white blood cells and preferably refers to monocytes and lymphocytes (B, T and NK cells), either in combination or individually. Thus, it should be understood that the term leucocyte may also be used herein so as to refer to individual species of leucocytes such as T-cells only.

The term “biologically active fragment thereof” refers to peptides and polypeptides that are derived from said TGAP7 protein and that are capable of effecting the same or similar activity or at least one of said activities of TGAP7-α [see SEQ ID NO: 3 or 4] or TGAP7-β [see SEQ ID NO: 1 or 2]

In accordance with the present invention, a gene induced in the early stage of T-cell activation has been identified by examining mRNA expression in alloactivated human lymphocytes. Differential display-reverse transcription PCR analysis revealed a 450 bp cDNA fragment which was upregulated 24 h after allostimulation of a human T-cell line; see Example 1. The corresponding complete cDNAs named TGAP7-α (comprising 5965 bp/predicted 1783 amino acids) and TGAP7-β (comprising 6034 bp/predicted 1804 amino acids) amino acid protein share 100% homology with the putative GAP-protein E6TPI as described by Gao (1999), MCB19, 733-744. TGAP7 mRNA is expressed in a variety of human tissues with various expression levels; see appended Example 2. Strictly, TGAP7 is expressed in all immuno tissues with highest expression levels in spleen, peripheral blood, lymph nodes, appendix, thymus, fetal liver and bone marrow cells. TGAP7 is expected to function in cell proliferation and differentiation events during T-cell and/or general leucocyte activation. More importantly, it has been found in accordance with the present invention that TGAP7 is probably essential in the differentiation events from quiescent to activated leucocytes, particular T-cells or other leucocytes, like B-cells, NK-cells or monocytes after stimulation by allo-/autoantigens or in response to antigens, like bacterial or viral antigens. This could be demonstrated by antisense expression experiments with antisense polynucleotides derived from the TGAP7 cDNA. From this experiment, it can be concluded that TGAP7 plays an essential role in activation of immune responses at an early stage.

From the above it is evident that the nucleotide sequencers) depicted in SEQ ID NOs. 1, 3, 5 or 6 encode(s) a novel class of immune response modulating proteins which were previously described as a putative GAP-protein (see Gao (1999), loc. cit.). By the provision of these nucleotide sequences it is now possible to isolate identical or similar polynucleotides which code for proteins with the biological, immunological activity of TGAP7 from other species or organisms. Said nucleotide sequences may be employed, in accordance with this invention, in the pharmaceutical compositions, uses and/or methods described herein. Well-established approaches for the identification and isolation of such related sequences are, for example, the isolation from genomic or cDNA libraries using the complete or part of the disclosed sequence as a probe or the amplification of corresponding polynucleotides by polymerase chain reaction using specific primers. Thus, the invention also relates to pharmaceutical compositions comprising polynucleotides which hybridize to the above described polynucleotides and differ at one or more positions in comparison to these as long as they encode a TGAP7 protein as defined above. Such molecules comprise those which are changed, for example, by deletion(s), insertion(s), alteration(s) or any other modification known in the art in comparison to the above described polynucleotides either alone or in combination. Methods for introducing such modifications in the polynucleotides of the invention are well-known to the person skilled in the art; see, e.g., Sambrook et al. (Molecular cloning; A Laboratory Manual, Second Edition, Cold Spring Harbor Laboratory Press, Cold Spring Harbor N.Y. (1989)). The invention also relates to polynucleotides the nucleotide sequence of which differs from the nucleotide sequence of any of the above-described polynucleotides due to the degeneracy of the genetic code.

The term “hybridizing” is understood as referring to conventional hybridization conditions, preferably such as hybridization in 50%formamide/6×SSC/0.1%SDS/100 μg/ml ssDNA, in which temperatures for hybridization are-above 37° C. and temperatures for washing in 0.1×SSC/0.1%SDS are above 55° C. Most preferably, the term “hybridizing” refers to stringent hybridization conditions, for example such as described in Sambrook, supra.

Particularly preferred are polynucleotides which share at least 70%, preferably at least 85%, more preferably 90-95%, and most preferably 96-99% sequence identity with one of the above-mentioned polynucleotides and have the same biological activity. Such polynucleotides also comprise those which are altered, for example by nucleotide deletion(s), insertion(s), substitution(s), addition(s), and/or recombination(s) and/or any other modification(s) known in the art either alone or in combination in comparison to the above-described polynucleotides. Methods for introducing such modifications in the nucleotide sequence of the polynucleotide of the invention are well known to the person skilled in the art. Thus, the pharmaceutical composition(s), use(s) and method(s) of the present invention may comprise any polynucleotide that can be derived from the above described polynucleotides by way of genetic engineering and that encode upon expression a TGAP7 protein or a biologically active fragment thereof.

It is also immediately evident to the person skilled in the art that regulatory sequences may be added to the polynucleotide as defined herein and employed in the pharmaceutical composition, uses and/or methods of the invention. For example, promoters, transcriptional enhancers and/or sequences which allow for induced expression of the polynucleotide of the invention may be employed. A suitable inducible system is for example tetracycline-regulated gene expression as described, e.g., by Gossen and Bujard (Proc. Natl. Acad. Sci. USA 89 (1992), 5547-5551) and Gossen et al. (Trends Biotech. 12 (1994), 58-62).

In a further embodiment, the invention relates to a pharmaceutical composition comprising nucleic acid molecules of at least 15 nucleotides in length hybridizing specifically with a polynucleotide as described above or with a complementary strand thereof. Such nucleic acid molecules are often referred to as antisense (nucleic acid) molecules although they do not have to be necessarily “antisense” to the coding region of a gene but can have the same polarity as the cDNA. Repression of gene expression can still take place for example because of triple helix effects and/or sense supression, etc. Specific hybridization occurs preferably under stringent conditions and implies no or very little cross-hybridization with nucleotide sequences encoding no or substantially different proteins. Such nucleic acid molecules may be used as probes and/or for the control of gene expression. Nucleic acid probe technology is well known to those skilled in the art who will readily appreciate that such probes may vary in length. Preferred are nucleic acid probes of 17 to 35 nucleotides in length. Of course, it may also be appropriate to use nucleic acids of up to 100 and more nucleotides in length. Said nucleic acid probes are particularly useful for various pharmaceutical and/or diagnostic applications. On the one hand, they may be used as PCR primers for amplification of polynucleotides encoding TGAP7 proteins and/or its homologues and may, thereby, serve as useful diagnostic tools. Another application is the use as a hybridization probe to identify polynucleotides hybridizing to the polynucleotides encoding TGAP7 by homology screening of genomic DNA libraries. Nucleic acid molecules employed in this preferred embodiment of the invention which are complementary to a polynucleotide as described above may also be used for repression of expression of a gene comprising such a polynucleotide, for example due to an antisense or triple helix effect or for the construction of appropriate ribozymes (see, e.g., EP-A1 0 291 533, EP-A1 0 321 201, EP-A2 0 360 257) which specifically cleave the (pre)-mRNA of a gene comprising a polynucleotide as described herein above. Selection of appropriate target sites and corresponding ribozymes can be done as described for example in Steinecke, Ribozymes, Methods in Cell Biology 50, Galbraith et al. eds Academic Press, Inc. (1995), 449-460. Standard methods relating to antisense technology have also been described (Melani, Cancer Res. 51 (1991), 2897-2901). Said antisense or triple helix effect as well as the construction of relevant ribozymes is/are particularly useful in pharmaceutical compositions to be employed for the suppression of the immune system, e.g., in autoimmune diseases, for the treatment of rejection events during or after transplantation, etc. Furthermore, the person skilled in the art is well aware that it is also possible to label such a nucleic acid probe with an appropriate marker for specific (inter alia, diagnostic) applications, such as for the detection of the presence of a polynucleotide as described herein above in a sample derived from an organism.

The above described nucleic acid molecules may either be DNA or RNA or a hybrid thereof. Furthermore, said nucleic acid molecule may contain, for example, thioester bonds and/or nucleotide analogues, commonly used in oligonucleotide anti-sense approaches. Said modifications may be useful for the stabilization of the nucleic acid molecule against endo- and/or exonucleases in the cell. Said nucleic acid molecules may be transcribed by an appropriate vector containing a chimeric gene which allows for the transcription of said nucleic acid molecule in the cell. Such nucleic acid molecules may further contain ribozyme sequences as described above.

In this respect, it is also to be understood that the polynucleotide to be used in the invention can be employed for “gene targeting” and/or “gene replacement”, for restoring a mutant gene or for creating a mutant gene via homologous recombination; see for example Mouellic, Proc. Natl. Acad. Sci. USA, 87 (1990), 4712-4716; Joyner, Gene Targeting, A Practical Approach, Oxford University Press.

In a particular preferred embodiment of the present invention, the pharmaceutical composition comprising polynucleotides as defined herein above may be employed in vaccination approaches. Such vaccination approaches may be, inter alia, useful in prevention or treatment of malignant diseases, for example in the prevention or therapy of tumors of the hematopoietic system. Vaccination approaches employing nucleic acid molecules are well known in the art and are described, inter alia, in Leither, Vaccine 18 (2000), 765-777.

In a preferred embodiment said nucleic acid molecules to be employed in the pharmaceutical composition(s), uses and/or methods of the invention are labeled. Said labels may comprise radiolabels or fluorescence labels. In another preferred embodiment said nucleic acid molecules may be used for the suppression of TGAP7 expression. Particularly preferred in this embodiment are the above described hybridizing nucleic acid molecules.

The polynucleotide as employed in accordance with this invention and encoding the above described TGAP7 protein or (a) biologically active fragment(s) thereof may be, e.g., DNA, cDNA, RNA or synthetically produced DNA or RNA or a recombinantly produced chimeric nucleic acid molecule comprising any of those polynucleotides either alone or in combination. Preferably the polynucleotides including antisense molecules are part of a vector. Such vectors may comprise further genes such as marker genes which allow for the selection of said vector in a suitable host cell and under suitable conditions. Preferably, the polynucleotides are operatively linked to expression control sequences allowing expression in prokaryotic or eukaryotic cells. Expression of said polynucleotide comprises transcription of the polynucleotide into a translatable mRNA. Regulatory elements ensuring expression in eukaryotic cells, preferably mammalian cells, are well known to those skilled in the art. They usually comprise regulatory sequences ensuring initiation of transcription and optionally poly-A signals ensuring termination of transcription and stabilization of the transcript. Additional regulatory elements may include transcriptional as well as translational enhancers, and/or naturally-associated or heterologous promoter regions. Possible regulatory elements permitting expression in prokaryotic host cells comprise, e.g., the P_{L}, lac, trp or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. Beside elements which are responsible for the initiation of transcription such regulatory elements may also comprise transcription termination signals, such as the SV40-poly-A site or the tk-poly-A site, downstream of the polynucleotide. Furthermore, depending on the expression system used leader sequences capable of directing the polypeptide to a cellular compartment or secreting it into the medium may be added to the coding sequence of the polynucleotide of the invention and are well known in the art. The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the heterologous sequence can encode a fusion protein including an C- or N-terminal identification peptide imparting desired characteristics, e.g., stabilization or simplified purification of expressed recombinant product. In this context, suitable expression vectors are known in the art such as Okayama-Berg cDNA expression vector pcDV1 (Pharmacia), pCDM8, pRc/CMV, pcDNA1, pcDNA3 (In-vitrogene), or pSPORT1 (GIBCO BRL).

Preferably, the expression control sequences will be eukaryotic promoter systems in vectors capable of transforming or transfecting eukaryotic host cells, but control sequences for prokaryotic hosts may also be used. Once the vector has been incorporated into the appropriate host, the host is maintained under conditions suitable for high level expression of the nucleotide sequences, and, as desired, the collection and purification of the protein of the invention may follow; see, e.g., the appended examples. In one preferred embodiment of the present invention antisense constructs are made based on the polynucleotide encoding TGAP7 (or (a) biologically active fragment(s) thereof) and combined with an appropriate expression control sequence.

In accordance with the above, the present invention relates to pharmaceutical compositions comprising (a) vector(s), particularly (a) plasmid(s), cosmid(s), virus(es) and bacteriophage(s) used conventionally in genetic engineering that comprise a polynucleotide encoding a TGAP7 protein and/or (a) functional fragment(s) thereof (as defined herein above). Methods which are well known to those skilled in the art can be used to construct recombinant vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1989). Alternatively, the polynucleotides and vectors to be employed in accordance with this invention can be reconstituted into liposomes for delivery to target-cells preferably to cells of the immune system. The here described vectors containing the polynucleotides described herein above can be transferred into the host-cell by well-known methods, which vary depending on the type of cellular host. For example, calcium chloride transfection is commonly utilized for prokaryotic cells, whereas calcium phosphate treatment or electroporation may be used for other cellular hosts; see Sambrook, supra.

In a more preferred embodiment, the present invention provides for a pharmaceutical composition comprising a vector as defined herein above, wherein said polynucleotide or nucleic acid molecule is operably linked to regulatory sequences allowing for the transcription and, optionally, expression of said nucleic acid molecules.

In a still further embodiment, the present invention relates to a pharmaceutical composition comprising a cell, preferably a host cell, containing the polynucleotide or vector described above. Preferably, said cell is a eukaryotic, most preferably a mammalian cell if therapeutic uses are envisaged. Of course, yeast and less preferred prokaryotic, e.g., bacterial cells may serve as well, in particular if the produced protein is used as a diagnostic means or if said protein is employed in methods as described herein above.

The polynucleotide or vector described herein which is present in the host cell may either be integrated into the genome of the host-cell or it may be maintained extrachromosomally.

The term “prokaryotic” is meant to include all bacteria which can be transformed or transfected with a DNA or RNA molecules for the expression of a protein of the invention. Prokaryotic hosts may include gram negative as well as gram positive bacteria such as, for example, E. coli, S. typhimurium, Serratia marcescens and Bacillus subtilis. The term “eukaryotic” is meant to include yeast, higher plant, insect and preferably mammalian cells. Depending upon the host employed in a recombinant production procedure, the protein encoded by the polynucleotide of the present invention may be glycosylated or may be non-glycosylated. TGAP7 proteins as employed in accordance with the present invention may also include an initial methionine amino acid residue. A polynucleotide as described herein can be used to transform or transfect the host using any of the techniques commonly known to those of ordinary skill in the art. Furthermore, methods for preparing fused, operably linked genes and expressing them in, e.g., mammalian cells and bacteria are well-known in the art (Sambrook, Molecular Cloning: A Laboratory Manual, Cold Spring Harbor Laboratory, Cold Spring Harbor, N.Y., 1989). The genetic constructs and methods described therein can be utilized for expression of the TGAP7 protein in eukaryotic or prokaryotic hosts. In general, expression vectors containing promoter sequences which facilitate the efficient transcription of the inserted polynucleotide are used in connection with the host. The expression vector typically contains an origin of replication, a promoter, and a terminator, as well as specific genes which are capable of providing phenotypic selection of the transformed cells. Furthermore, transgenic animals, preferably mammals, comprising nucleic acid molecules/polynucleotides as defined herein may be used for the large scale production of the TGAP7 protein and/or for the large scale production of pharmaceutical compositions described herein.

Alternatively, an animal, preferably mammalian cell naturally having a polynucleotide described herein present in its genome can be used and modified such that said cell expresses the endogenous gene corresponding to the polynucleotide described herein above under the control of an heterologous promoter. The introduction of the heterologous promoter which does not naturally control the expression of the polynucleotide of the invention can be done according to standard methods, see supra. Suitable promoter include those mentioned hereinbefore.

In this context, it should be mentioned that a method for the production of a TGAP7 protein or a biologically active fragment thereof may comprise:

(a) culturing a host described herein above under conditions allowing for the expression of the protein; or

(b) in vitro translation of the polynucleotide encoding TGAP7 and/or a biologically active fragment thereof;

and recovering the protein (or a fragment thereof) produced in (a) or (b).

The transformed hosts can be grown in fermentors and cultured according to techniques known in the art to achieve optimal cell growth. The TGAP7 protein and/or biologically active fragments thereof to be employed in pharmaceutical compositions, uses and/or methods of this invention can then be isolated from the growth medium, cellular lysates, or cellular membrane fractions. Once expressed, the protein of the present invention can be purified according to standard procedures of the art, including ammonium sulfate precipitation, affinity columns, column chromatography, gel electrophoresis and the like; see, Scopes, “Protein Purification”, Springer-Verlag, N.Y. (1982). Substantially pure proteins of at least about 90 to 95% homogeneity are preferred, and 98 to 99% or more homogeneity are most preferred, for pharmaceutical uses. Once purified, partially or to homogeneity as desired, the proteins may then be used therapeutically (including extracorporeally) or in developing and performing assay procedures.

Hence, in a still further embodiment, the present invention relates to a pharmaceutical composition comprising a TGAP7 protein or a biologically active fragment thereof encoded by the polynucleotide described herein above or produced by a method of as above. It will be apparent to those skilled in the art that the TGAP7 protein or a (biologically active) fragment thereof can be further coupled to other moieties as described above for, e.g., drug targeting and imaging applications, i.e. for pharmaceutical and/or diagnostic uses. Such coupling may be conducted chemically after expression of the protein to site of attachment or the coupling product may be engineered into the protein of the invention at the DNA level. The DNAs are then expressed in a suitable host system, and the expressed proteins are collected and renatured, if necessary. TGAP7 proteins/polypeptides may by particularly useful in (a) pharmaceutical setting(s) where specific leucocyte activation should be controlled. As mentioned herein below, specific overexpression of TGAP7 proteins or (biologically acitve) fragments thereof may be obtained by gene therapeutic approaches. As documented in the appended examples, TGAP7 expression is induced by activation of cells of the immune system. Without being bound by theory, it is therefore envisaged that one function of TGAP7 is the control of cell activation events in the immune system.

Furthermore, the provision of the TGAP7 protein as described herein above enables the production of TGAP7 specific antibodies. In this respect, hybridoma technology enables production of cell lines secreting antibody to essentially any desired substance that produces an immune response. RNA encoding the light and heavy chains of the immunoglobulin can then be obtained from the cytoplasm of the hybridoma. The 5′ end portion of the mRNA can be used to prepare cDNA to be inserted into an expression vector. The DNA encoding the antibody or its immunoglobulin chains can subsequently be expressed in cells, preferably mammalian cells.

Depending on the host-cell, renaturation techniques may be required to attain proper conformation of the antibody. If necessary, point substitutions seeking to optimize binding may be made in the DNA using conventional cassette mutagenesis or other protein engineering methodology such as is disclosed herein.

Thus, the present invention also relates to a pharmaceutical composition comprising an antibody specifically recognizing TGAP7 protein or (a) fragment(s) (peptides, polypeptides) thereof.

In a preferred embodiment of the invention, said antibody comprised in said pharmaceutical composition is a monoclonal antibody, a polyclonal antibody, a single chain antibody, humanized antibody, a xenogeneic antibody or fragment thereof that specifically binds said peptide or polypeptide also including bispecific antibody, synthetic antibody, antibody fragment, such as Fab, Fv or scFv fragments etc., or a chemically modified derivative of any of these. Monoclonal antibodies can be prepared, for example, by the techniques as originally described in Köhler and Milstein, Nature 256 (1975), 495, and Galfré, Meth. Enzymol. 73 (1981), 3, which comprise the fusion of mouse myeloma cells to spleen cells derived from immunized mammals with modifications developed by the art. Furthermore, antibodies or fragments thereof to the aforementioned peptides can be obtained by using methods which are described, e.g., in Harlow and Lane “Antibodies, A Laboratory Manual”, CSH Press, Cold Spring Harbor, 1988. When derivatives of said antibodies are obtained by the phage display technique, surface plasmon resonance as employed in the BIAcore system can be used to increase the efficiency of phage antibodies which bind to an epitope of the peptide or polypeptide of the invention (Schier, Human Antibodies Hybridomas 7 (1996), 97-105; Malmborg, J. Immunol. Methods 183 (1995), 7-13). The production of chimeric antibodies is described, for example, in WO89/09622. Methods for the production of humanized antibodies are described in, e.g., EP-A1 0 239 400 and WO90/07861. A further source of antibodies to be utilized in accordance with the present invention are so-called xenogenic antibodies. The general principle for the production of xenogenic antibodies such as human antibodies in mice is described in, e.g., WO 91/10741, WO 94/02602, WO 96/34096 and WO 96/33735. Antibodies to be employed in accordance with the invention or their corresponding immunoglobulin chain(s) can be further modified using conventional techniques known in the art, for example, by using amino acid deletion(s), insertion(s), substitution(s), addition(s), and/or recombination(s) and/or any other modification(s) known in the art either alone or in combination. Methods for introducing such modifications in the DNA sequence underlying the amino acid sequence of an immunoglobulin chain are well known to the person skilled in the art; see, e.g., Sambrook, Molecular Cloning A Laboratory Manual, Cold Spring Harbor Laboratory (1989) N.Y. It is particularly preferred that the here described pharmaceutical compositions comprise antibodies/antibody contructs which may be employed in intracellular settings. Such antibody constructs/antibodies are well known in the art and are, inter alia, described in Lener (2000), Eur. J. Biochem. 267, 1196-1205, who described intracellular antibodies against p21 ras.

In a still further embodiment, the present invention relates to a cell that has been modified to express a TGAP7 protein or an antibody as described herein. This embodiment may be well suited for, e.g., restoring B and/or T-cell responsiveness to an antigen, in particular if the antibody of the invention capable of stimulating T-cell proliferation is expressed in a form suitable to be presented on the cell surface.

The present invention furthermore relates to a pharmaceutical composition comprising an antisense construct capable of inhibiting the expression of a polynucleotide encoding TGAP7 (and/or (a) biologically active fragment(s) thereof) as defined herein above. As illustration in the appended example (Example 3) such antisense constructs/oligonucleotides are particularly useful in the down regulation of leucocyte/lymphocyte responses/activations. Therefore, the here described pharmaceutical compositions comprising (specific) antisense constructs which are capable of inhibiting the expression of TGAP7 may be particularly useful in the treatment and/or prevention of pathological or medical situations where an immunoactivation is not desired. These situations comprise, but are not limited to, treatment of acute and chronic rejections of allo- and xeno- (organ)transplants or bone marrow transplantations, inflammation processes and/or allergies. The use of antisense oligonucleotides/constructs is well known in the art and described, inter alia, in Irizawa (1995), Clin. Exp. Immunology 100, 383-389 or Boeve (1994), J. Leucocyte Biol. 55, 169-174. Methods and computer programs for the preparation and rational selection of antisense oligonucleotide sequences are described in the prior art; see for example Smith, Eur. J. Pharm. Sci. 11 (2000), 191-198; Toschi, Methods 22 (2000), 261-269; Sohail, Adv. Drug Deliv. Rev. 44 (2000), 23-34; Moulton, J. Comput. Biol. 7 (2000), 277-292. These procedures comprise how to find optimal hybridization sites, and on how to select sequences that bind to for example TGAP7 mRNA. These methods can include the more empirical testing of large numbers of mRNA complementary sequences to the more systematic techniques, i.e. RNase H mapping, use of combinatorial arrays and prediction of secondary structure of mRNA by computational methods. Structures that bind to structured RNA, i.e. aptastrucs and tethered oligonucleotide probes, and foldback triplex-forming oligonucleotides can also be employed for the purpose of the present invention. Relating to selection of antisense sequences by aid of computational analysis, valuable www addresses are given in the above-identified prior art.

Secondary structure prediction and in vitro accessibility of mRNA as tools in the selection of target sites for ribozymes is described for example in Amarzguioui, Nucleic Acids Res. 28 (2000), 4113-4124. Minimising the secondary structure of DNA targets by incorporation of a modified deoxynucleoside and implications for nucleic acid analysis by hybridisation is described in Nguyen, Nucleic Acids Res. 28 (2000), 3904-3909. Preferably, the antisense molecules comprise at least 14 or 15, more preferably about 17 to 20 or more, and most preferably about at least 20, 25 or 30 or more consecutive nucleotides (including nucleotide analogs) of or complementary to any one of the above described polynucleotides encoding TGAP7 or corresponding genomic sequences, including 5′- and 3′-untranslated regions, introns, transcriptional regulatory sequences and the like. In a preferred embodiment the antisense molecule comprises said at least 14 or 15 nucleotides complementary to any one of SEQ ID NOS: 1, 3 and 5.

In yet another embodiment the present invention relates to the use of the pharmaceutical composition(s) described herein for use in cell or organ transplantation, for the treatment of autoimmune, allergic or infectious diseases or for the treatment of tumors.

An example for the use of the pharmaceutical composition of the invention for improving allograft or xenograft tolerance is described with respect to administration of an LFA-3 and CD2 binding protein, respectively, in WO93/06852.

The pharmaceutical composition of the present invention may further comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical or intradermal administration. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 to 1000 μg (or of nucleic acid for expression or for inhibition of expression in this range); however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Progress can be monitored by periodic assessment. Dosages will vary but a preferred dosage for intravenous administration of DNA is from approximately 10⁶ to 10¹² copies of the DNA molecule. The compositions of the invention may be administered locally or systemically. Administration will generally be parenterally, e.g., intravenously; DNA may also be administered directly to the target site, e.g., by biolistic delivery to an internal or external target site or by catheter to a site in an artery. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents such as T-cell, B-cell, NK-cell or monocyte costimulatory molecules or cytokines known in the art, or their inhibitors or activators depending on the intended use of the pharmaceutical composition.

Furthermore, it is envisaged by the present invention that the various polynucleotides and vectors encoding the above described peptides or polypeptides are administered either alone or in any combination using standard vectors and/or gene delivery systems, and optionally together with a pharmaceutically acceptable carrier or excipient. For example, the polynucleotide of the invention can be used alone or as part of a vector to express the (poly)peptide described herein in cells, for, e.g., gene therapy or diagnostics of diseases related to disorders of the immune system. The polynucleotides or vectors described herein are introduced into the cells which in turn produce the TGAP7 protein (or (a) fragment(s) thereof). Subsequent to administration, said polynucleotides or vectors may be stably integrated into the genome of the subject. On the other hand, viral vectors may be used which are specific for certain cells or tissues and persist in said cells. Suitable pharmaceutical carriers and excipients are well known in the art. The pharmaceutical compositions prepared according to the invention can be used for the prevention or treatment or delaying of different kinds of diseases, which are related to leucocyte, lymphocyte and/or monocyte related immunodeficiencies and malignancies such as multiple myeloma, T-, B-cell leukemia, infectious diseases related to T-, B-, NK-cell and monocyte proliferation, immune activation in rejection of transplants, autoimmune disorders, Allergy.

In another embodiment the present invention relates to a diagnostic composition comprising any one of the above described proteins, antibodies, (poly)peptides, polynucleotides, vectors or cells, and optionally suitable means for detection. The (poly)peptides and antibodies described above are, for example, suited for use in immunoassays in which they can be utilized in liquid phase or bound to a solid phase carrier. Examples of immunoassays which can utilize said (poly)peptide are competitive and non-competitive immunoassays in either a direct or indirect format. Examples of such immunoassays are the radioimmunoassay (RIA), the sandwich (immunometric assay) and the Western blot assay. The (poly)peptides and antibodies can be bound to many different carriers and used to isolate cells specifically bound to said polypeptides. Examples of well-known carriers include glass, polystyrene, polyvinyl chloride, polypropylene, polyethylene, polycarbonate, dextran, nylon, amyloses, natural and modified celluloses, polyacrylamides, agaroses, and magnetite. The nature of the carrier can be either soluble or insoluble for the purposes of the invention.

There are many different labels and methods of labeling known to those of ordinary skill in the art. Examples of the types of labels which can be used in the present invention include enzymes, radioisotopes, colloidal metals, fluorescent compounds, chemiluminescent compounds, and bioluminescent compounds. The here described diagnostic compositions are particularly useful for the detection of an activated status of the immune system, in particular to detect activation of T-cells, B-cells, NK-cells and/or monocytes.

Said diagnostic compositions may also be used for methods for detecting expression of a polynucleotide encoding TGAP7 (or its homologues) by detecting the presence of mRNA coding for a TGAP7 protein which comprises obtaining mRNA from a cell and contacting the mRNA so obtained with a probe comprising a nucleic acid molecule of at least 15 nucleotides capable of specifically hybridizing with a polynucleotide encoding TGAP7 (or its homologues) under suitable hybridizing conditions (see also supra), detecting the presence of mRNA hybridized to the probe, and thereby detecting the expression of the TGAP7 protein (or its homologues) by the cell.

Furthermore, the invention comprises methods of detecting the presence of a TGAP7 protein in a sample, for example, a cell sample, which comprises obtaining a cell sample from a subject, contacting said sample with one of the aforementioned antibodies under conditions permitting binding of the antibody to the TGAP7 protein, and detecting the presence of the antibody so bound, for example, using immuno assay techniques such as radio-immunoassay or enzyme-immunoassay. Furthermore, one skilled in the art may specifically detect and distinguish polypeptides which are functional TGAP7 proteins from mutated forms which have lost or altered their leucocyte (T-cell, B-cell, etc.) stimulatory activity by using an antibody which either specifically recognizes a (poly)peptide which has TGAP7 activity but does not recognize an inactive form thereof or which specifically recognizes an inactive form but not the corresponding polypeptide having TGAP7 activity. The antibodies as described in the present invention may also be used in affinity chromatography for purifying the TGAP7 protein or above described (poly)peptides and isolating them from various sources. Said purified proteins/(poly)peptides may be employed in the pharmaceutical compositions, uses and/or methods of the present invention.

The invention also encompasses a method for diagnosing in a subject a predisposition (susceptibility) to a disorder associated with the expression of a TGAP7 allele which comprises isolating DNA from victims of the the disorder associated with the under-or over-expression of a TGAP7 protein; digesting the isolated DNA with at least one restriction enzyme; electrophoretically separating the resulting DNA fragments on a sizing gel; contacting the resulting gel with a nucleic acid probe as described above capable of specifically hybridizing to DNA encoding a TGAP7 protein and labeled with a detectable marker; detecting labeled bands on the gel which have hybridized to the labeled probe to create a band pattern specific to the DNA of victims of the disorder associated with the expression of a TGAP7 protein; preparing the subject's DNA according to the above-mentioned steps to produce detectable labeled bands on a gel; and comparing the band pattern specific to the DNA of victims of the disorder associated with the expression of a TGAP7 protein and the subject's DNA to determine whether the patterns are the same or different and to diagnose thereby predisposition to the disorder if the patterns are the same. The detectable markers of the present invention may be labeled with commonly employed radioactive labels, such as, for example, ³²P and ³⁵S, although other labels such as biotin or mercury as well as those described above may be employed as well. Various methods well-known to the person skilled in the art may be used to label the detectable markers. For example, DNA sequences and RNA sequences may be labeled with ³²P or ³⁵S using the random primer method. Once a suitable detectable marker has been obtained, various methods well-known to the person skilled in the art may be employed for contacting the detectable marker with the sample of interest. For example, DNA-DNA, RNA-RNA and DNA-RNA hybridizations may be performed using standard procedures. Various methods for the detection of nucleic acids are well-known in the art, e.g., Southern and northern blotting, PCR, primer extension and the like. Furthermore, the mRNA, cRNA, cDNA or genomic DNA obtained from the subject may be sequenced to identify mutations which may be characteristic fingerprints of TGAP7 mutations in disorders associated with the expression of TGAP7 or mutated versions thereof. The present invention further comprises methods, wherein such a fingerprint may be generated by RFLPs of DNA or RNA obtained from the subject, optionally the DNA or RNA may be amplified prior to analysis, the methods of which are well known in the art. RNA fingerprints may be performed by, for example, digesting an RNA sample obtained from the subject with a suitable RNA-Enzyme, for example RNase T₁, RNase T₂ or the like or a ribozyme and, for example, electrophoretically separating and detecting the RNA fragments on PAGE as described above or in the appended examples.

In another embodiment, the present invention relates to a pharmaceutical composition comprising an agent which stimulates a leucocyte through the TGAP7 protein as described herein, and optionally a pharmaceutically acceptable carrier. As is immediately evident to the person skilled in the art, the provision of the TGAP7 as an immunomodulating molecule opens up the way of alternative approaches for leucocytes stimulation and treating corresponding diseases. The agent that stimulates the proliferation of leucocytes or lymphocytes through the TGAP7 protein is expected to markedly enhance the proliferation of leucocytes or lymphocytes of, e.g., (activated) T-cells and thus is capable of augmenting the immune response. Examples for this type of “vaccine” are described, e.g., in WO91/11194 and in the literature, e.g., referred to above. The agents to be employed in accordance with the present invention usually specifically bind and/or interact to TGAP7 protein in order to exert their effect. Such agents can be identified in accordance with a method of the invention described below. Such agents also comprise promoters which can be inserted in front of the coding region of the TGAP7 protein encoding gene, e.g., via gene transfer and homologous recombination in the 5′ untranslated region of the gene, see also supra. Such promoter may be regulated and thus permit the controlled expression of the TGAP7 protein in certain cells.

Therefore, in a further aspect the present invention relates to a method for identifying a binding partner to a TGAP7 polypeptide comprising:

(a) contacting a TGAP7 polypeptide (protein) of the invention with a compound to be screened; and

(b) determining whether the compound effects an activity of the polypeptide (protein).

TGAP7 polypeptides may be used to screen for molecules that bind to TGAP7 or for molecules to which TGAP7 binds. The binding of TGAP7 and the molecule may activate (agonist), increase, inhibit (antagonist), or decrease activity of the TGAP7 or the molecule bound. Examples of such molecules include antibodies (including single-chain antibodies), oligonucleotides, proteins (e.g., receptors), or small molecules.

Preferably, the molecule is closely related to the natural binding partner of TGAP7, e.g., a fragment of the binding partner, or a natural substrate, a “ligand”, a structural or functional mimetic; see, e.g., Coligan, Current Protocols in Immunology 1(2) (1991); Chapter 5. Similarly, the molecule can be closely related to the natural binding partner(s) with which TGAP7 interacts, or at least, a fragment of said binding and/or interaction partner capable of being bound by TGAP7 (e.g., active site). In either case, the molecule can be rationally designed using known techniques; see also infra. (A) potential binding partner(s) of TGAP7 is/are G-protein interacting molecule(s).

Preferably, the screening for these molecules involves producing appropriate cells which express TGAP7, either as a secreted protein or as a protein in or on the cell membrane. Preferred cells include cells from mammals, yeast, Drosophila, or E. coli. Cells expressing TGAP7 (or cell membrane(s) containing the expressed polypeptide) are then preferably contacted with a test compound potentially containing the molecule to observe binding, stimulation, or inhibition of activity of either TGAP7 or the molecule.

The assay may simply test binding of a candidate compound to TGAP7, wherein binding is detected by a label, or in an assay involving competition with a labeled competitor. Further, the assay may test whether the candidate compound results in a signal generated by binding to TGAP7.

Alternatively, the assay can be carried out using cell-free preparations, polypeptide/molecule affixed to a solid support, chemical libraries, or natural product mixtures. The assay may also simply comprise the steps of mixing a candidate compound with a solution containing TGAP7, measuring TGAP7/molecule activity or binding, and comparing the TGAP7/molecule activity or binding to a standard. Preferably, an ELISA assay can measure TGAP7 level or activity in a sample (e.g., biological sample) using a monoclonal or polyclonal antibody. The antibody can measure TGAP7 level or activity by either binding, directly or indirectly, to TGAP7 or by competing with TGAP7 for a substrate.

All of these above assays can be used as diagnostic or prognostic markers. The molecules discovered using these assays can be used to treat disease or to bring about a particular result in a patient (e.g., increase of immune response) by activating or inhibiting the TGAP7/molecule. Moreover, the assays can discover agents which may inhibit or enhance the production of TGAP7 from suitably manipulated cells or tissues.

Therefore, the invention includes a method of identifying compounds which bind to TGAP7 comprising the steps of:

(a) incubating a candidate binding compound with TGAP7; and

(b) determining if binding has occurred.

Moreover, the invention includes a method of identifying agonists/antagonists comprising the steps of:

(a) incubating a candidate compound with TGAP7;

(b) assaying a biological activity as described above, and

(c) determining if a biological activity of TGAP7 has been altered.

As mentioned hereinbefore, the polynucleotides encoding TGAP7 (or (a) fragment(s) thereof) and polypeptides representing TGAP7 (or (a) fragment(s) thereof) provide a basis for the development of mimetic compounds that may be inhibitors or activators of TGAP7 or their encoding genes. It will be appreciated that the present invention also provides cell based screening methods that allow a high-throughput-screening (HTS) of compounds that may be candidates for such inhibitors and activators.

Furthermore, the invention relates to a method for identifying leucocyte activating or co-stimulating compounds or for identifying inhibitors of leucocyte activation and stimulation comprising

(a) culturing leucocytes, lymphocytes or monocytes in the presence of the TGAP7 protein, (poly)peptide, antibody, cell and/or the antisense construct described above and, optionally, in the presence of a component capable of providing a detectable signal in response to leucocyte proliferation/activation, with a compound to be screened under conditions permitting interaction of the compound with the TGAP7 protein, (poly)peptide, antibody or cell(s); and

(b) detecting the presence or absence of a signal generated from the interaction of the compound with the cells.

The term “compound” in the method of the invention includes a single substance or a plurality of substances which may or may not be identical.

Said compound(s) may be comprised in, for example, samples, e.g., cell extracts from, e.g., plants, animals or microorganisms. Furthermore, said compounds may be known in the art but hitherto not known to be capable of inhibiting proliferation of leucocytes or not known to be useful as an immune response costimulatory factor, respectively. The plurality of compounds may be, e.g., added to a simple in vitro, to the culture medium or injected into the cell.

If a sample containing (a) compound(s) is identified in the method of the invention, then it is either possible to isolate the compound from the original sample identified as containing the compound, in question or one can further subdivide the original sample, for example, if it consists of a plurality of different compounds, so as to reduce the number of different substances per sample and repeat the method with the subdivisions of the original sample. It can then be determined whether said sample or compound displays the desired properties by methods known in the art such as described herein and in the appended examples. Depending on the complexity of the samples, the steps described above can be performed several times, preferably until the sample identified according to the method of the invention only comprises a limited number of or only one substance(s). Preferably said sample comprises substances of similar chemical and/or physical properties, and most preferably said substances are identical. The methods of the present invention can be easily performed and designed by the person skilled in the art, for example in accordance with other cell based assays described in the prior art (see, e.g., EP-A-0 403 506) or by using and modifying the methods as described in the appended examples. Furthermore, the person skilled in the art will readily recognize which further compounds and/or cells may be used in order to perform the methods of the invention, for example, B cells, interleukins, or enzymes, if necessary, that, e.g., convert a certain compound into the precursor which in turn stimulates or suppresses lymphocyte or monocyte activation or that provide for (co)stimulatory signals. Such adaptation of the method of the invention is well within the skill of the person skilled in the art and can be performed without undue experimentation.

Compounds which can be used in accordance with the method of the present invention include peptides, proteins, nucleic acids including cDNA expression libraries, antibodies, small organic compounds, ligands, peptidomimetics, PNAs and the like. Said compounds can also be functional derivatives or analogues of known leucocyte, lymphocyte (B-, T- or NK-cell) or monocyte activators or inhibitors. Methods for the preparation of chemical derivatives and analogues are well known to those skilled in the art and are described in, for example, Beilstein, Handbook of Organic Chemistry, Springer edition New York Inc., 175 Fifth Avenue, New York, N.Y. 10010 U.S.A. and Organic Synthesis, Wiley, New York, USA. Furthermore, said derivatives and analogues can be tested for their effects according to methods known in the art or as described, for example, in the appended examples. Furthermore, peptidomimetics and/or computer aided design of appropriate activators or inhibitors of leucocytes, lymphocytes, monocytes (like T-cell, B-cell, NK-cell) activation can be used, for example, according to the methods described below. Appropriate computer programs can be used for the identification of interactive sites of a putative inhibitor and the TGAP7 protein (or its biologically active fragment(s)) by computer assistant searches for complementary structural motifs (Fassina, Immunomethods 5 (1994), 114-120). Further appropriate computer systems for the computer aided design of protein and peptides are described in the prior art, for example, in Berry, Biochem. Soc. Trans. 22 (1994), 1033-1036; Wodak, Ann. N. Y. Acad. Sci. 501 (1987), 1-13; Pabo, Biochemistry 25 (1986), 5987-5991. The results obtained from the above-described computer analysis can be used in combination with the method of the invention for, e.g., optimizing known leucocyte activators or inhibitors. Appropriate peptidomimetics can also be identified by the synthesis of peptidomimetic combinatorial libraries through successive chemical modification and testing the resulting compounds, e.g., according to the methods described herein and in the appended examples. Methods for the generation and use of peptidomimetic combinatorial libraries are described in the prior art, for example in Ostresh, Methods in Enzymology 267 (1996), 220-234 and Dorner, Bioorg. Med. Chem. 4 (1996), 709-715. Furthermore, the three-dimensional and/or crystallographic structure of inhibitors or activators of leucocyte stimulation can be used for the design of peptidomimetic inhibitors or activators of leucocyte activation to be tested in the method of the invention (Rose, Biochemistry 35 (1996), 12933-12944; Rutenber, Bioorg. Med. Chem. 4 (1996), 1545-1558).

In summary, the present invention provides methods for identifying compounds which are capable of modulating immune responses. Accordingly compounds identified in accordance with the method of the present invention to be inhibitors and activators, respectively, of immune response are also within the scope of the present invention.

Compounds found to enhance leucocyte proliferation may be used in the treatment of cancer or infections and related diseases. In addition, it may also be possible to specifically inhibit viral diseases, thereby preventing viral infection or viral spread. Compounds identified as suppressors of leucocyte proliferation can be used, e.g., for treating skin conditions (see, e.g., WO93/06866) or in allogenic or xenogenic cell or organ transplantation in order to avoid graft rejection; see also supra. The compounds identified or obtained according to the method of the present invention are thus expected to be very useful in diagnostic and in particular for therapeutic applications.

Hence, in a further embodiment the invention relates to a method for the production of a pharmaceutical composition comprising formulating and optionally synthesizing the compound identified in step (b) or (c) of the above described methods of the invention in a pharmaceutically acceptable form. Hence, the present invention generally relates to a method of making a therapeutic agent comprising synthesizing the proteins, (poly)peptides, polynucleotides, vectors, antibodies or compounds according to the invention in an amount sufficient to provide said agent in a therapeutically effective amount to the patient. Methods for synthesizing these agents are well known in the art and are described, e.g. above.

The therapeutically useful compounds identified according to the method of the invention may be administered to a patient by any appropriate method for the particular compound, e.g., orally, intravenously, parenterally, transdermally, transmucosally, or by surgery or implantation (e.g., with the compound being in the form of a solid or semi-solid biologically compatible and resorbable matrix) at or near the site where the effect of the compound is desired. Therapeutic doses are determined to be appropriate by one skilled in the art, see also supra.

Such useful compounds can be for example transacting factors which bind to the TGAP7 protein described herein. Identification of transacting factors can be carried out using standard methods in the art (see, e.g., Sambrook, supra, and Ausubel, supra). To determine whether a protein binds to the TGAP7 protein, standard native gel-shift analyses can be carried out. In order to identify a transacting factor which binds to the TGAP7 protein, the polypeptides and peptides described in this invention can be used as an affinity reagent in standard protein purification methods, or as a probe for screening an expression library. Once the transacting factor is identified, modulation of its binding to the TGAP7 protein as described herein can be pursued, beginning with, for example, screening for inhibitors against the binding of the transacting factor to the TGAP7 protein. Activation or repression of TGAP7 specific genes could then be achieved in subjects by applying the transacting factor (or its inhibitor) or the gene encoding it, e.g., in a vector described in the embodiments hereinbefore. In addition, if the active form of the transacting factor is a dimer, dominant-negative mutants of the transacting factor could be made in order to inhibit its activity. Furthermore, upon identification of the transacting factor, further components in the pathway leading to activation (e.g. signal transduction) or repression of a gene encoding the TGAP7 protein described herein can then be identified. Modulation of the activities of these components can then be pursued, in order to develop additional drugs and methods for modulating the expression or activity of the TGAP7 protein.

Beside the above described possibilities to use the polynucleotides according to the invention for gene therapy and their use to identify homologous molecules, the described polynucleotides may also be used for several other applications, for example, for the identification of nucleic acid molecules which encode proteins which interact with the TGAP7 protein described above. This can be achieved by assays well known in the art, for example, as described in Scofield (Science 274 (1996), 2063-2065) by use of the so-called yeast “two-hybrid system”. In this system the (poly)peptide encoded by the polynucleotides according to the invention or a smaller part thereof is linked to the DNA-binding domain of the GAL4 transcription factor. A yeast strain expressing this fusion protein and comprising a lacZ reporter gene driven by an appropriate promoter, which is recognized by the GAL4 transcription factor, is transformed with a library of cDNAs which will express animal, preferably mammal proteins or peptides thereof fused to an activation domain. Thus, if a peptide encoded by one of the cDNAs is able to interact with the fusion protein comprising a (poly)peptide of the invention, the complex is able to direct expression of the reporter gene. In this way the polynucleotide according to the invention and the encoded peptide can be used to identify peptides and proteins interacting with TGAP7 proteins. Other methods for identifying compounds which interact with the TGAP7 protein according to the invention or nucleic acid molecules encoding such molecules are, for example, the in vitro screening with the phage display system as well as filter binding assays or “real time” measuring of interaction using, for example, the BIAcore apparatus (Pharmacia); see references cited supra.

Furthermore, the present invention relates to the use of the polynucleotide, the nucleic acid molecule, the vectors, peptides, polypeptides, antibodies and cells described herein as well as compounds identified in accordance with a method of the invention described hereinabove for the preparation of a composition for diagnosing and/or the treatment of acute and chronic diseases involving T-cell activation and associated with Th1 and Th2 immune response, for the treatment of acute and chronic rejection of allo-and xeno organ transplants and bone marrow transplantation, for the treatment of rheumatoid arthritis, lupus erythramatodes, multiple sklerosis, encephalitis, vasculitis, diabetes mellitus, pancreatitis, gastritis, thyroiditis, for the treatment of disorders (inter alia malignant disorders) of T-, B- or NK-cells, for the treatment of asthma, lepramatosis, Helicobacter pylori associated gastritis or for the treatment of skin tumors, adrenal tumors or lung tumors, wound healing, growth disorders, inflammatory and/or infectious diseases. It is particularly preferred that the polynucleotide encoding TGAP7 (or (a) fragment(s) thereof) or the antibody as defined herein above is employed for the detection of leucocyte activation and/or for the treatment of diseases linked to leucocyte activation.

As described before, the present invention for the first time provided a novel route of therapeutic intervention via modulating, preferably inhibiting the activity of the above-described TGAP7 protein. Therefore, the present invention generally relates to a method for the treatment of a disease, disorder or condition as described above which comprises administering to a cell, tissue, organ or subject an effective amount of a compound capable of suppressing TGAP7 activity. Preferably, said suppressing of TGAP7 activity results in inhibiting of the proliferation of PHA activated T-cell-lymphocytes. This can be tested e.g. according to the method described in Example 3. Compounds that may be used for the above-described methods include those identified by the methods of the present invention and comprise for example TGAP7 antisense molecules, an anti-TGAP7 antibodies, peptides or peptide mimetics of TGAP7 protein, ligands, substrates or binding partners of TGAP7.

The polynucleotides, vectors, cells, proteins, (poly)peptides, antibodies, inhibitors, activators, pharmaceutical and diagnosis compositions, uses described herein above and methods of the invention can be used for the treatment of all kinds of diseases hitherto unknown as being related to or dependent on the modulation of TGAP7. The pharmaceutical compositions, methods and uses of the present invention may be desirably employed in humans, although animal treatment is also encompassed by the methods and uses described herein.

These and other embodiments are disclosed and encompassed by the description and Examples of the present invention. Further literature concerning any one of the antibodies, methods, uses and compounds to be employed in accordance with the present invention may be retrieved from public libraries and databases, using for example electronic devices. For example the public database “Medline” may be utilized which is available on the Internet, for example under http://www.ncbi.nlm.nih.gov/PubMed/medline.html. Further databases and addresses, such as http://www.ncbi.nlm.nih.gov/, http://www.infobiogen.fr/, http://www.fmi.ch/biology/research_tools.html, http://www.tigr.org/, are known to the person skilled in the art and can also be obtained using, e.g., http://www.lycos.com. An overview of patent information in biotechnology and a survey of relevant sources of patent information useful for retrospective searching and for current awareness is given in Berks, TIBTECH 12 (1994), 352-364.

### BRIEF DESCRIPTION OF DRAWINGS

This disclosure may best be understood in conjunction with the accompanying drawings, incorporated herein by references, which show:

FIG. 1:

Sequence comparison of amino acids between TGAP7 alpha and beta.

FIG. 2a:

Expression of the 6 and 9 kb mRNAs of TGAP7 in different tissues of the immune system.

FIG. 2b:

TGAP7 is induced after allostimulation of human lymphocytes (MLC 24). Furthermore, TGAP7 is expressed in EBV-transformed B-cell lines as well as in Jurkat cells (Jr, Ja).

FIG. 3:

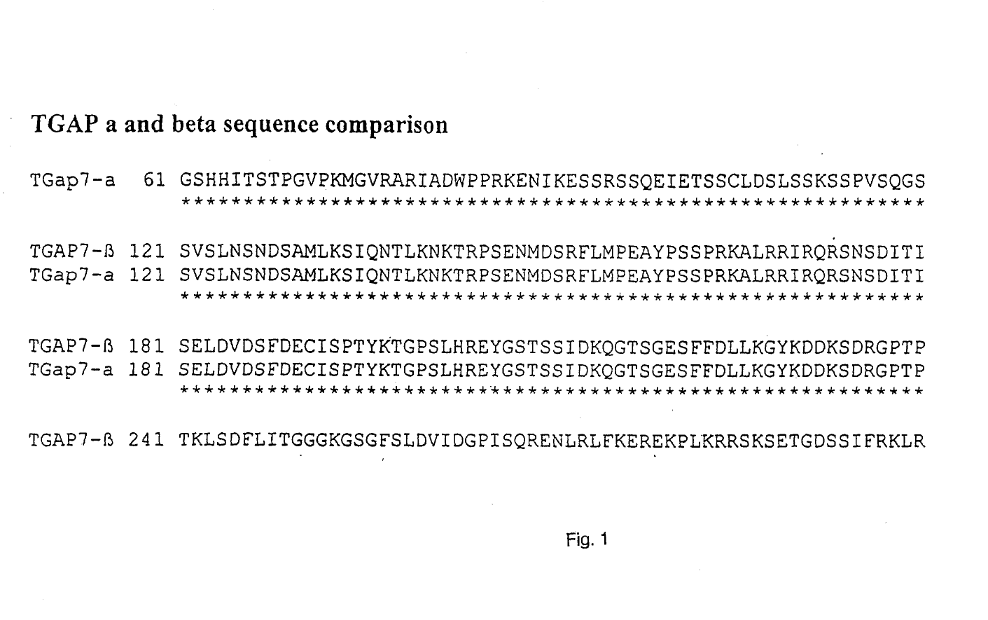

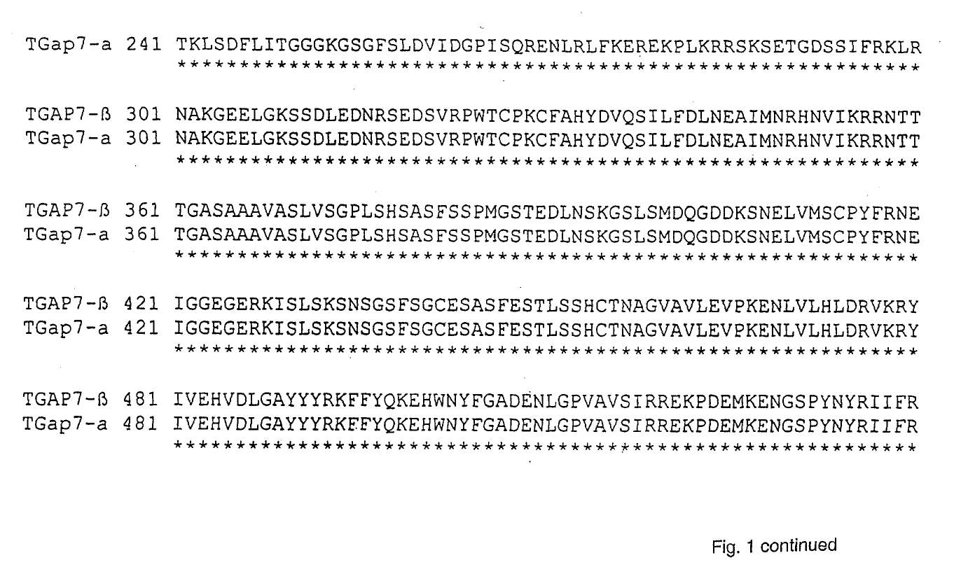

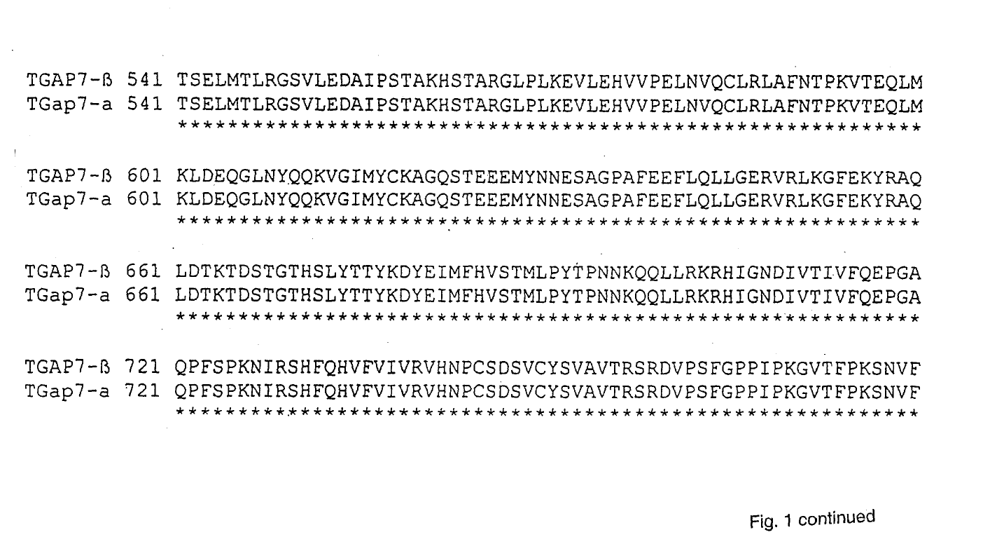

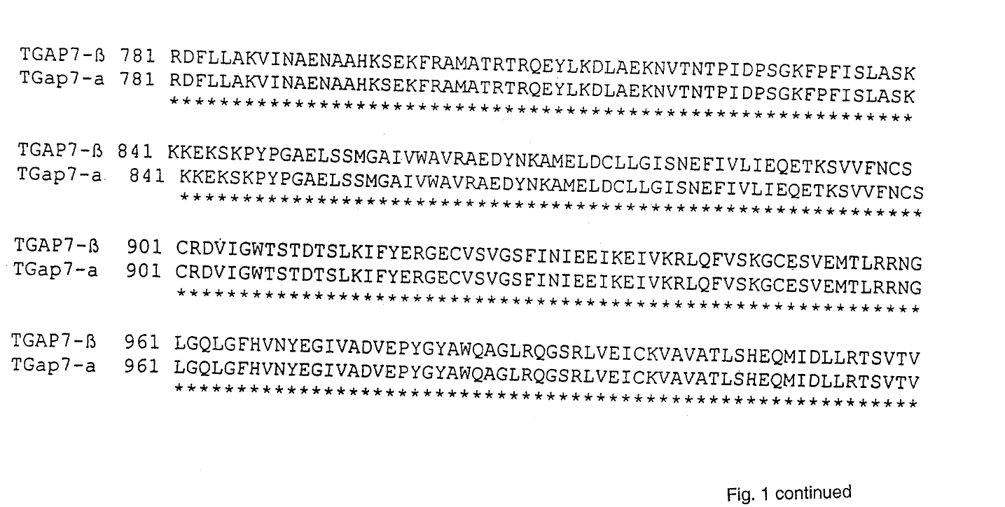

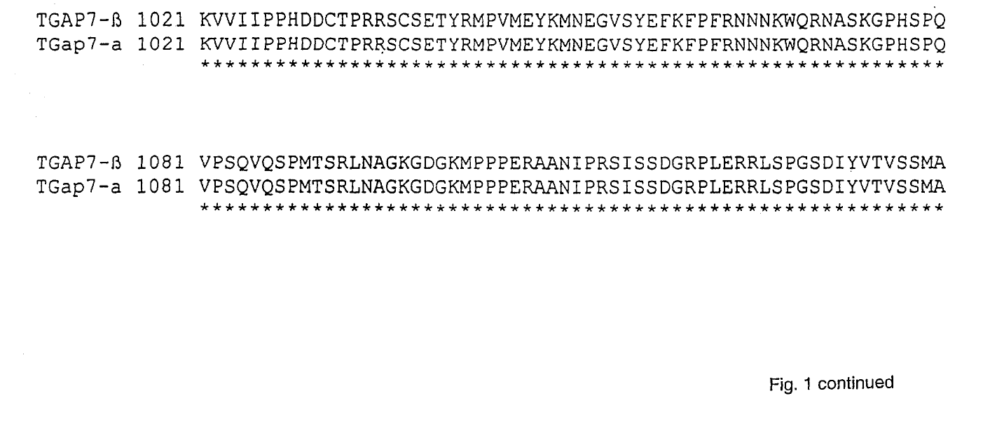

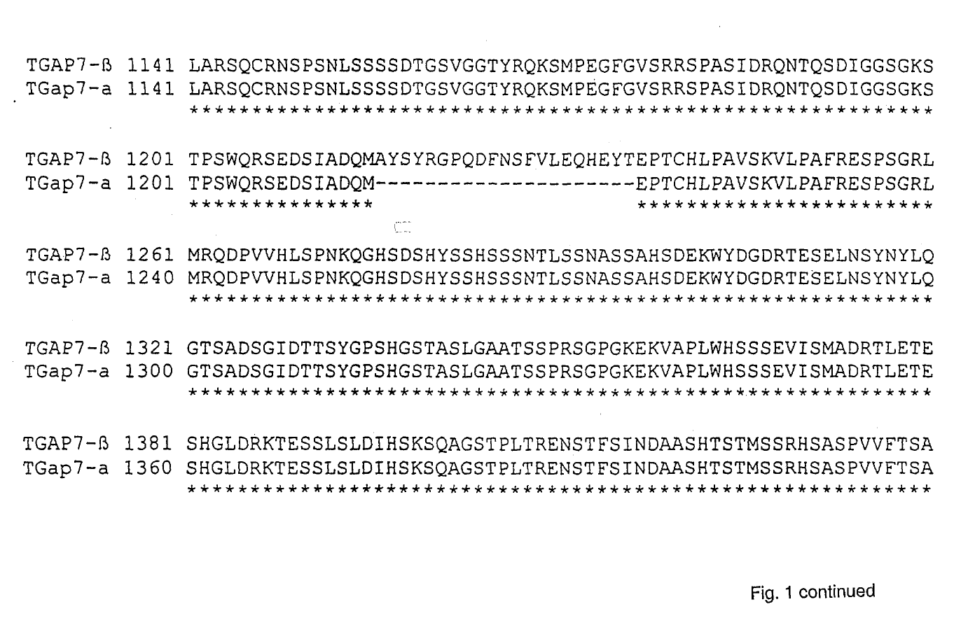

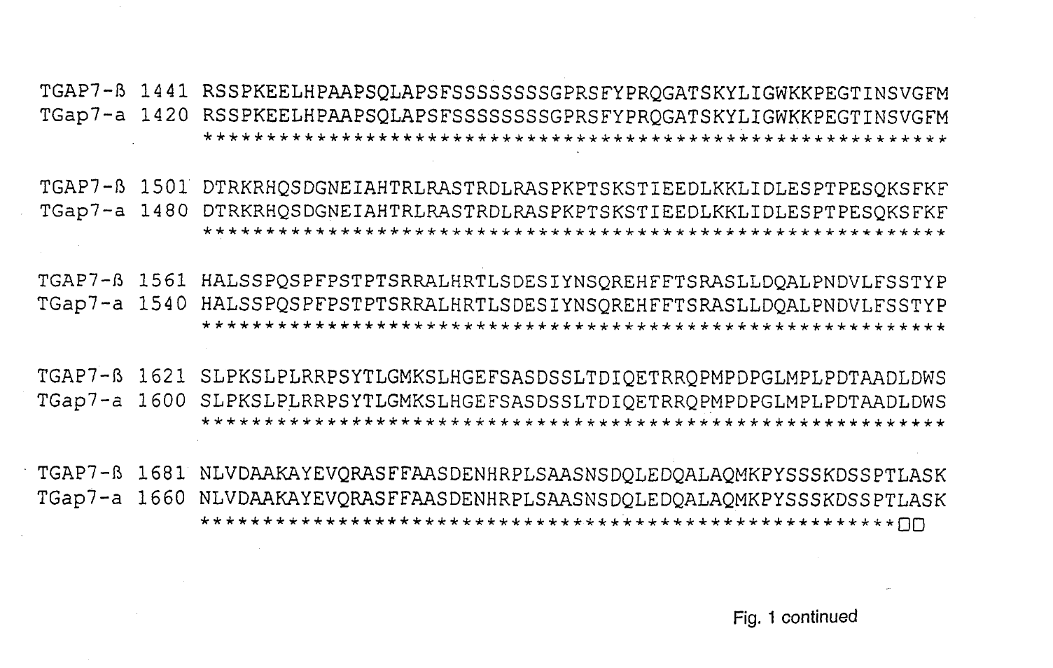

Antiproliferative effects of TGAP7 antisense oligonucleotides on PHA activated T-cells in vitro. Intense proliferative inhibition of T-GAP-anti-Sense 2 (5 M) in contrast to the control-oligo and the further control without oligo-addition.

A better understanding of the present invention and of its many advantages will be had from the following examples, given by way of illustration.

### EXAMPLE 1

### Cloning of a Novel cDNA TGAP7, That is Differentially Expressed in Allo- and Mitogen Activated Human T Cells

To identify novel genes induced during the early stages of T cell activation in response to alloantigens, differential display RT-PCR analysis of mRNA expression was performed at time 0 and 24 h after initiation of a human mixed lymphocyte culture (MLR). In conformance with institutional policies regarding human experimentation, peripheral blood lymphocytes (PBLs, “mixed lymphcoytes”) were isolated from healthy human volunteers using standard Ficoll centrifugation methods and diluted into RPMI containing 10% fetal calf serum. Responder PBLs were stimulated with equal numbers of irradiated (3000 rad, 13 min) stimulator PBLs. Cells were co-cultured for 24 h in tissue flasks at an initial concentration of 10⁶ cells/ml for RNA isolation. For mitogen stimulation, cells were cultured in PHA or ConA (1 mg/l) in culture flasks at 37° C. at an initial concentration of 10 Million cells/ml for 24 h prior to isolation of RNA. Total RNA was isolated from MLR at 0 and 24 h using the RNAzol B method (Tel-Test, Inc) and differential display was performed as described previously (Kojima et al., 1996 [10]). Briefly, 2 μg of total RNA was reverse transcribed using an oligo-dT primer and 200 U MMLV reverse transcriptase (Gibco/BRL). A 40 cycle PCR amplification with a total volume of 10 μl was performed by using 1 μg of cDNA, 1.25 mM MgCl₂, 50 mM KCl, 10 mM Tris-HCl (pH 8.3), 2.5 nM primer, 5 μCi ³⁵S-dATP, and 0.3 U Taq polymerase. The primers for the PCR amplification were: 5′- GACGGAACAGCTTC -3′ [SEQ ID NO: 7] and 5′- TGCGTCTGGTTCT-3′ [SEQ ID NO: 8].

The PCR products were stored at 4° C. and separated by electrophoresis in 6% polyacrylamide-urea gels, transferred to filter paper, dried, and autoradiographed. The differentially expressed cDNA fragment was excised from the gel, eluted, reamplifed, cloned into pBluescriptSK⁺ plasmid, and sequenced. Homology searches were performed using BLAST at NCBI. Alignments were performed using Geneworks 2.1.1.

Results:

DDRTPCR analysis (Ref: Utku (1998) Prevention of acute allograft rejection by antibody targeting of TIRC7, a novel T cell membrane protein, Immunity, 509-518) of alloactivated human lymphocytes revealed a 450 bp fragment which was used to isolate the full-length cDNA from human T cell library, Said human T-cell cDNA library (obtained from Clontech) was screened by using the 450 bp TGAP7 cDNA fragment which revealed several clones. TGAP7 alpha (5965 bp) and TGAP7 beta (6034 bp) show identity to recently published cDNA clones obtained by yeast two hybrid screening (Gao (1999), loc. cit.)

### EXAMPLE 2

### Expression Studies for TGAP7

Northern blots containing RNA from various human tissues were purchased from Clontech. Northern blots were probed with TGAP7 cDNA. Overnight hybridizations were performed with ³²P labeled cDNA probes (10⁶ cpm/μl) at 42° C. in 40% formamide, 10% dextran sulfate, 4×SSC, 7 mM Tris (pH 7.6), 0.8×Denhardt's solution, 0.02 mg/ml salmon sperm DNA, and 10% SDS. Blots were washed twice in 2×SSC and 0.1% SDS for 20 min at room temperature, once at 65° C. in 0.2×SSC, 0.1% SDS and autoradiographed at −80° C.

Northern blots were prepared with 7-10 μg of total RNA as described previously (Kojima et al., 1996 [10]). Northern blots were probed with the full-length TGAP7 cDNA. Overnight hybridizations were performed with ³²P labeled cDNA probes (10⁶ cpm/μl) at 42° C. in 40% formamide, 10% dextran sulfate, 4×SSC, 7 mM Tris (pH 7.6), 0.8×Denhardt's solution, 0.02 mg/ml salmon sperm DNA, and 10% SDS. Blots were washed twice in 2×SSC and 0.1% SDS for 20 min at room temperature, once at 65° C. in 0.2×SSC, 0.1% SDS and autoradiographed at −80° C.

Results:

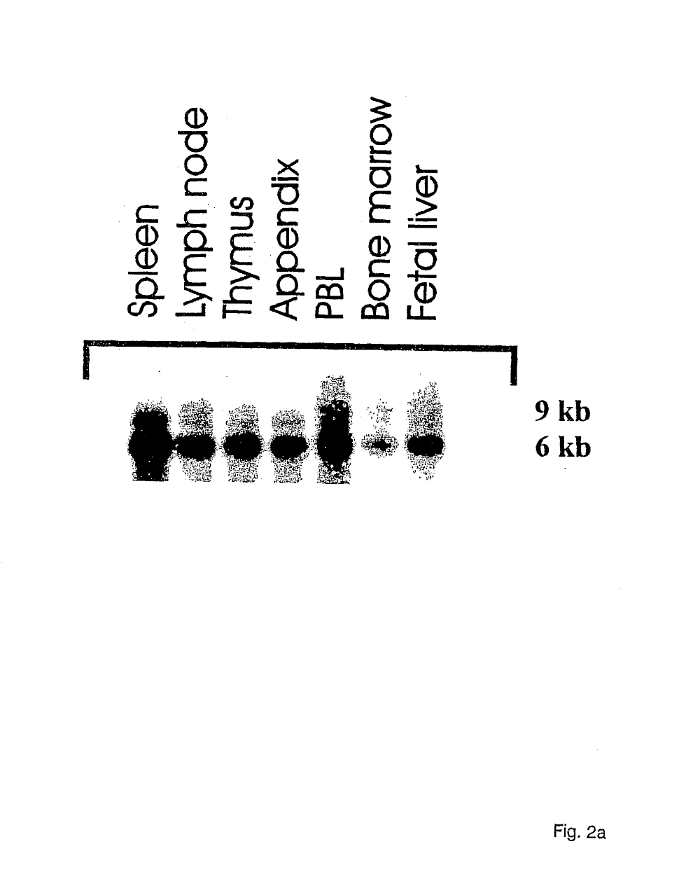

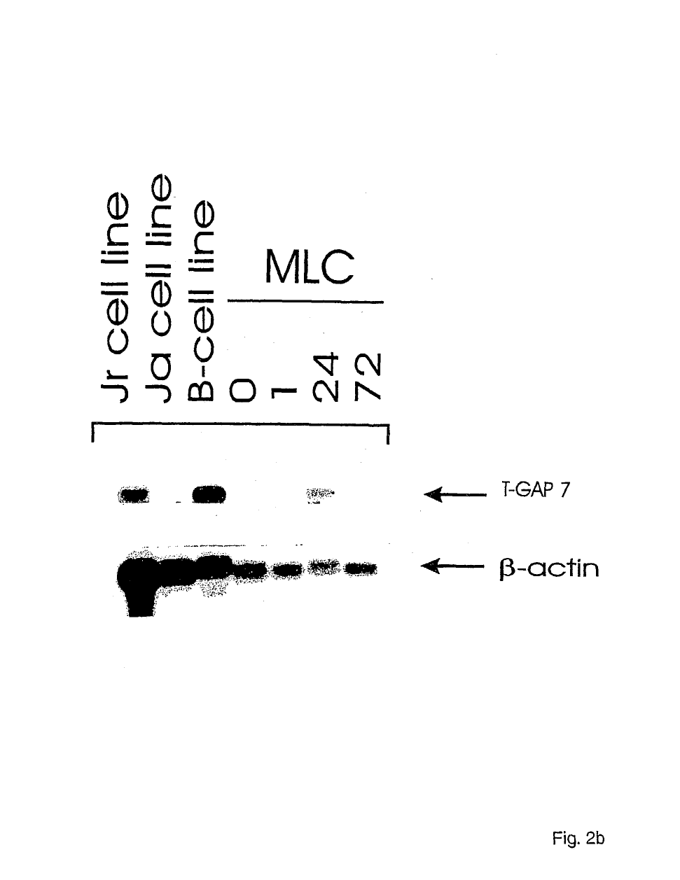

A TGAP7 specific cDNA probe detected 6 and 9 kb transcripts. Northern analysis revealed that TGAP7 6 kb cDNA is expressed in all immune tissues and exhibits highest levels of mRNA expression in spleen, and peripheral blood, followed by lymph nodes, appendix, thymus, and fetal liver and bone marrow cells, respectively. (FIG. 2a).

To determine the expression kinetics of TGAP7, Northern blot analysis of total RNA from alloantigen activated lymphocytes was performed.

A TGAP7 specific cDNA probe detected only the 6 kb transcript which resulted after alloactivation of PBL in a 10-fold upregulation of TGAP7 expression at 24 h. The mRNA is significantly present in EBV transformed B cells and Jurkat cells, respectively (FIG. 2b).

### EXAMPLE 3

### Effects of TGAP7 Specific Antisense Oligonucleotids in T-cell Proliferation

Peripheral blood lymphocytes (PBMC) were isolated from human volunteers using standard Ficoll centrifugation methods and diluted into RPMI 1640 containing 10% FKS. Number of cells was determined with Neubauer hemocytometer.

PBMC were exposed to phytohemagglutinin (PHA) (1 μg/ml). 20.000 cells in a final volume of 0,1 ml of complete RPMI were added to individual wells of a 96-well microtiter plate.

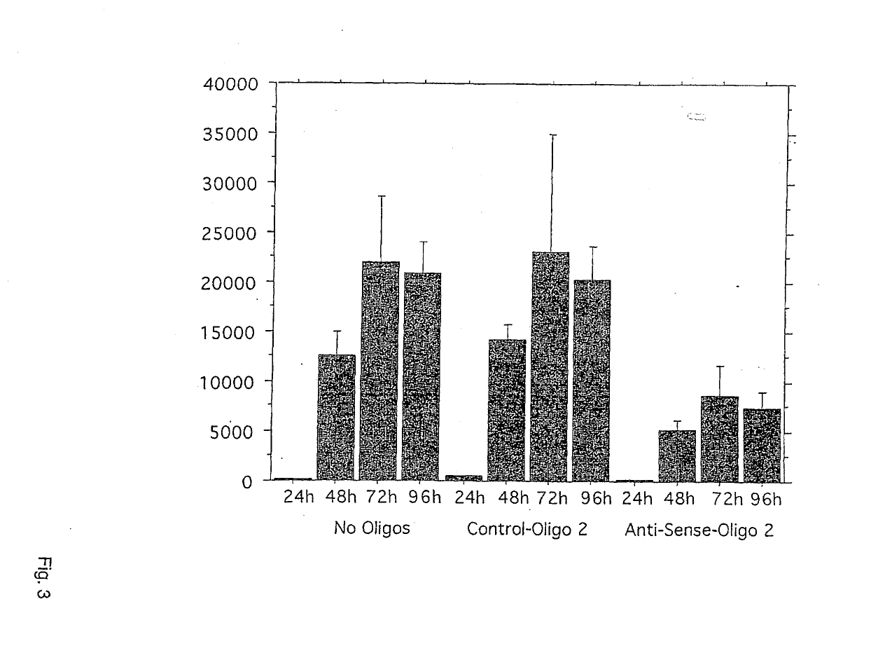

To study influence of TGAP7-antisense oligonucleotides on proliferative response of PHA-stimulated lymphocytes, cells were incubated in the presence and absence of an antisense oligonucleotide A2, a sense oligonucleotide (A1) complementary to A2 and two control oligonucleotides (control oligo C1 and C2) in a concentration of 5 μM oligos (diluted into 1×TE-Puffer, pH 7.2). The 96-well plates were incubated at 37° C. and 5% CO₂ 24 hr, 48 hr, 72 hr, 96 hr and 168 hr, pulsed with 1 μCi of [³H] thymidine per well and harvested 6 hrs later. Incorporated radioactivity was determined in a scintillation counter and the datas were evaluated with StatView (FIG. 3).

Results:

The creation of immune response was analyzed in the presence and absence of TGAP7 specific antisense oligonucleotides in in vitro cultures of human lymphocytes such as in mixed lymphocyte culture including T, B, NK and monocytes and mitogen activated cells. As shown in FIG. 2, the results demonstrate a significant downregulation of immune response to alloantigen and mitogens in the presence of TGAP7 specific antisense (A2), whereas sense oligonucleotide, Al, or other control oligonucleotides did not exhibit any immunomodulatory effect.

Those skilled in the art will recognize, or be able to ascertain using no more than routine experimentation, many equivalents to the specific embodiments of the invention described herein. Such equivalents are intended to be encompassed by the following claims.

### REFERENCES

[1] G. R. Crabtree, Contingent genetic regulatory events in T lymphocyte activation, Science 243 (1989) 355-361.

[2] C. H. June, Signal transduction in T-cells, Curr. Opin. Immunol. 3 (1991) 287-293.

[3] R. H. Schwartz, Costimulation of T lymphocytes: the role of CD28, CTLA-4, and B7/BB1 in Interleukin-2 production and immunotherapy, Cell 71 (1992) 1065-1068.

[4] J. Banchereau, F. Bazan, D. Blanchard, F. Briere, J. Galizzi, C. van Kooten, Y. Liu, F. Rousset, S. Seeland, The CD40 antigen and its ligand, Annu. Rev. Immunol. 12 (1994) 881-922.

[5] D. J. Lenschow, T. Walunas, J. Bluestone, CD28/B7 system of T-cell costimulation, Annu. Rev. Immunol. 14 (1996) 233-258.

[6] P. Linsley, J. Ledbetter, The role of the CD28 receptor during T-cell responses to antigen, Annu. Rev. Immunol. 11 (1993) 191-212.

[7] A. Kupfer, S. L. Swain, S. J. Singer, The specific direct interaction of helper T-cells and antigen-presenting B cells. II. Reorientation of the microtubule organizing center and reorganization of the membrane-associated cytoskeleton inside the bound helper T-cells, J. Exp. Med. 165 (1987) 1565-1580.

[8] M. V. Parsey, G. K. Lewis, Actin polymerization and pseudopod reorganization accompany anti-CD3-induced growth arrest in Jurkat T-cells, J. Immunol. 151 (1993) 1881-1893.

[9] N. Selliah, W. H. Brooks, T. L. Roszman, Proteolytic cleavage of -actinin by calpain in T-cells stimulated with anti-CD3 monoclonal antibody, J. Immunol. 156 (1996) 3215-3221.

[10] R. Kojima, J. Randall, B. M. Brenner, S. R. Gullans, Osmotic stress protein 94 (Osp94): A new member of the Hsp110/SSE gene subfamily, J. Biol. Chem. 271 (1996) 12327-12332.

1. A pharmaceutical composition comprising a polynucleotide encoding a TGAP7 protein or a biologically active fragment thereof comprising a nucleic acid molecule selected from the group consisting of
(i) DNA sequences comprising a nucleotide sequence encoding the amino acid sequence depicted in SEQ ID NO: 2 or 4;
(ii) DNA sequences comprising the nucleotide sequence depicted in SEQ ID NO:1 or 3;
(iii) DNA sequences comprising a nucleotide sequence encoding a fragment or derivative of the protein encoded by the DNA sequence of (i) or (ii);
(iv) DNA sequences the complementary strand of which hybridizes with and which is at least 70% identical to the polynucleotide as defined in any one of (i) to (iii); and
(v) DNA sequences the nucleotide of which is degenerate to the nucleotide sequence of a DNA sequence of any one of (i) to (iv).

2. A pharmaceutical composition comprising a nucleic acid molecule of at least 15 nucleotides in length hybridizing specifically with a polynucleotide of claim 1 or with a complementary strand thereof

3. A pharmaceutical composition comprising a vector comprising the polynucleotide as defined in claim 1 or 2.

4. A pharmaceutical composition comprising a vector as defined in claim 3, wherein said polynucleotide or nucleic acid molecule is operably linked to regulatory sequences allowing for the transcription and optionally expression of said nucleic acid molecule.

5. A pharmaceutical composition comprising a host cell containing a polynucleotide as defined in claim 1 or 2 or a vector as defined in claim 3 or 4.

6. A pharmaceutical composition comprising a TGAP7 protein or (a) biologically active fragment(s) thereof encoded by the nucleic acid molecule as defined in claim 1.

7. A pharmaceutical composition comprising an antibody specifically recognizing the protein as defined in claim 6.

8. A pharmaceutical composition comprising an antisense construct capable of inhibiting the expression of a polynucleotide as defined in claim 1.

9. The pharmaceutical composition of any one of claims 1 to 8 for use in cell, tissue or organ transplantation, for the treatment of autoimmune, allergic or infectious diseases, or for the treatment of tumors.

10. A diagnostic composition comprising a polynucleotide as defined in claim 1 or 2, a vector as defined in claim 3 or 4, a cell as defined in claim 5, a protein as defined in claim 6, an antibody as defined in claim 7 or an antisense construct as defined in claim 8; and, optionally, suitable means for detection.

11. A method of diagnosing a pathological condition or a susceptibility to a pathological condition in a subject related to a disorder in the immune system comprising:
(a) determining the presence or absence of a mutation in the polynucleotide as defined in claim 1; and
(b) diagnosing a pathological condition or a susceptibility to a pathological condition based on the presence or absence of said mutation.

12. A method of diagnosing a pathological condition or a susceptibility to a pathological condition in a subject related to a disorder in the immune system comprising:
(a) determining the presence or amount of expression of the protein as defined in claim 6 in a biological sample; and
(b) diagnosing a pathological condition or a susceptibility to a pathological condition based on the presence or amount of expression of the protein.

13. A method for identifying a binding partner to a TGAP7 protein comprising:
(a) contacting a protein as defined in claim 6 with a compound to be screened; and
(b) determining whether the compound effects an activity of the protein.

14. A method for identifying leucocyte activating or co-stimulating compounds or for identifying inhibitors of leucocyte activation and stimulation comprising
(a) culturing leucocytes, lymphocytes or monocytes in the presence of the protein as defined in claim 6, the antibody as defined in claim 7, the cell as defined in claim 5 and/or the antisense construct as defined in claim 8; and, optionally, in the presence of a component capable of providing a detectable signal in response to leucocyte proliferation, with a compound to be screened under conditions permitting interaction of the compound with the (poly)peptide, antibody or cell(s); and
(b) detecting the presence or absence of a signal generated from the interaction of the compound with the cells.

15. A method for the production of a pharmaceutical compositions comprising the steps of the method of claims 13 and 14 and formulating and, optionally, synthesizing the compound identified in step (b) in a pharmaceutically acceptable form.

16. A method for determining the status of an immune response comprising analyzing the presence of the polynucleotide as defined in claim 1 or the protein as defined in claim 6.

17. Use of the polynucleotide as defined in claim 1 or 2, the vector as defined in claim 2 or 3, the protein as defined in claim 6, the cell as defined in claim 5, the antisense construct of claim 8 the antibody as defined in claim 9, or the compound identified according to the method of any one of claims 13 and 14 for the preparation of a composition for diagnosing or the treatment of acute and chronic diseases, involving T cell activation and Th1 and Th2 immune response, for the treatment of acute and chronic rejection of allo-and xeno organ transplants and bone marrow transplantation, for the treatment of rheumatoid arthritis, lupus erythramatodes, multiple sklerosis, encephalitis, vasculitis, diabetes mellitus, pancreatitis, gastritis, thyroiditis, for the treatment of maligne disorders of T, B or NK cells, for the treatment of asthma, lepramatosis, Helicobacter pylori associated gastritis or for the treatment of skin tumors, adrenal tumors or lung tumors, wound healing, growth disorders, inflammatory and/or infectious diseases.

18. Use of a polynucleotide as defined in claim 1 or the antibody as defined in claim 7 for the detection of leucocyte activation.

19. Use of claim 18, wherein said leucocyte is a B cell, T cell, NK cell and/or monocyte.

20. A method for the treatment of a disease, disorder or condition as defined in claim 17 which comprises administering to a cell, tissue, organ or subject an effective amount of a compound capable of suppressing TGAP7 activity.

21. The method of claim 20, wherein said compound is capable of inhibiting the proliferation of PHA activated T-cell-lymphocytes.

22. The method of claim 20 or 21 wherein said compound is a TGAP7 antisense molecule, an anti-TGAP7 antibody, a peptide or peptide mimetic of TGAP7 protein, or a ligand, substrate or binding partner of TGAP7.


SEQUENCE LISTING (8 sequences)

SEQ ID NO: 1 - DNA, 6028 bp, Homo sapiens
  CDS (349)..(5760)
ggtgtggacgttgtctaaatttcggtagccatggcacaagaatataagaaagcatgggattatggcaaccacagaatctcagtagtacaagttccattcagttttttctgaaagaaagccctctgttaaagtgaagcaaagaaactgttgtggattataacgtttagaagttccaatttttcagtgctttacaaataaagcatcatttaaccttttaaatgaaaaagattaagatctcatgcaactgttgtattttctggaagccattctccaaaagggaagtgcacatttaaaacacagatatgatggtccttgctgcagggatttaagtctacttgcttttacatcatgaccagcttgaaacggtcacagacagaaaggcctcttgccactgacagggcctctgttgttggcacagacggcacccccaaagtccacactgatgatttctacatgcggcgcttccggtcccaaaatggcagcttaggatcatcagttatggctcctgtaggacccccccgaagtgaaggttctcaccatataacctcaacccccggagtcccaaaaatgggggtaagggcaaggattgcagattggcccccaagaaaggaaaacataaaagaatctagccgttcaagccaggaaatagaaacctcaagttgccttgatagcctgtcctccaaaagcagtcctgtgagtcagggaagttctgttagcctcaattccaatgactcagccatgctgaaaagcatacagaacacgctgaaaaacaagacaagaccgtcggagaacatggactccagatttctcatgcctgaagcctaccccagctcccccagaaaagctcttcgcagaatacgccagcgaagcaacagtgatatcaccataagtgaacttgatgtggatagctttgatgaatgtatctcacctacatacaagactggaccatcactgcacagggaatatggtagcacatcttcaattgataaacagggaacatctggagaaagcttttttgatttgttaaagggctacaaagatgacaaatctgatcgaggtccaactccaaccaagctcagtgactttctcattactggtggtggcaagggttctggtttctctttggatgtaatagacgggcctatctcacagagagagaacctcaggctttttaaggaaagggaaaaaccactcaagcgacgttcaaaatctgaaactggagactcctctatttttcgtaaattgcgcaatgccaaaggtgaagaacttgggaagtcatcagatcttgaagataaccgatcagaagactctgtcaggccctggacatgtccaaagtgctttgcccactatgatgtccagagtatattatttgatttgaatgaggcaattatgaacaggcacaatgttattaagaggagaaacaccaccactggagcttccgcagctgccgtggcatccttggtctctggacctctgtctcattcagccagttttagctccccaatgggcagcacagaggacctgaattccaaaggaagcctcagcatggaccagggagatgataaaagcaatgagcttgtaatgagctgtccatattttcggaatgagataggtggagaaggggagaggaaaatcagcctttcaaaatcaaattctggctcctttagtggatgtgaaagtgcctcctttgagtctacccttagttcccattgcacaaatgcaggagtggcagtacttgaagtgcccaaggagaacttggtgttgcacctagatagagtgaaaagatacatcgtggaacacgtagatctgggtgcatactattatagaaaatttttctaccagaaggaacactggaactattttggggctgatgagaatcttggtccagtggctgtgagcattcgaagggaaaaaccagatgaaatgaaagaaaatggatctccgtacaactaccgaataatttttagaactagtgagctcatgacactgagaggttcggtcctggaggacgccattccgtcgacagccaagcactcgacagccagaggcctgcctctcaaagaagtgctggagcacgtggttcctgagctcaatgtccagtgcctgcggttggccttcaacacacccaaggtcacagagcagctcatgaaactggatgaacaagggctgaactaccagcagaaagtaggcatcatgtactgcaaagctggacagagcactgaagaagagatgtacaacaatgagtcagctggcccagcctttgaagaattccttcaactattgggagagcgagttcggctcaaaggatttgagaagtatcgagcacagcttgataccaaaactgactccactggaacccattctctgtacacaacatacaaagattatgaaattatgttccatgtttctaccatgctgccatacacacccaacaacaaacaacagctcctgaggaagcggcacattggaaatgatatcgtaacaattgttttccaagagcctggagcacagccattcagcccaaaaaacatccgatcccacttccagcacgttttcgtcatcgtcagggtgcacaatccgtgctctgacagtgtctgttatagtgtggctgttaccaggtccagagatgtgccttcctttgggcctcccattcctaaaggggtcactttccctaagtcaaatgtgttcagggacttccttttggcgaaagtgattaatgcagaaaatgctgctcataaatcggagaagtttcgggccatggcaactcggacccgccaggaatacctgaaagatctggcagaaaagaatgtcaccaacacccctatcgacccttctggcaagtttccgttcatctctctggcttccaagaagaaggaaaagtctaagccatatccaggagccgagctcagcagcatgggggccattgtatgggcagtccgggctgaagactacaacaaggccatggaactagactgccttttagggatctccaatgagttcattgtgctcattgaacaggaaacaaagagcgtggtcttcaattgttcctgtagagatgtgatagggtggacttcaactgacaccagcctcaaaatcttctatgaacgaggagaatgtgtttcagtgggtagttttattaacattgaggagatcaaagagattgtcaaaaggttgcagtttgtttcaaaaggctgtgaatcggtggagatgactctgcgaagaaatgggctaggacagcttggcttccatgtcaactatgagggcattgtggcggatgtggagccctacggttatgcctggcaggcagggctgaggcagggcagtcgcctggtggagatctgcaaggtggcggtagccactctgagccatgagcagatgatcgacctcctgagaacatctgtcacggtgaaggttgtcatcattcccccgcatgatgactgcaccccgcggaggagttgctctgaaacctaccgcatgccagtgatggagtacaaaatgaatgaaggtgtttcatacgaattcaagtttcccttccgaaataataacaagtggcagaggaacgccagcaaggggcctcattcacctcaagtcccgtcccaggtgcagagtcccatgacctcgcggctgaatgctggaaaaggagatgggaagatgcctcctccagaaagagccgccaacatccctcgaagcatctccagtgacgggcgcccactagagaggcggctgtctcctggttcggacatctatgtgacggtctcatccatggctttagcaagatcccagtgtcggaactctcctagcaacttgtcttcatccagtgatactggttctgtggggggcacttacaggcagaagtccatgcccgaagggtttggagtgagccgtagatccccagcctccattgacaggcagaacacccagtcagatattggtggcagcggaaaatccacgcctagctggcaaagaagtgaggatagcattgctgaccagatggcttacagttatagaggacctcaggatttcaattcttttgtcctcgagcagcatgaatatacagagccaacatgccatctcccagcagtatcaaaggtactgccagctttccgagagagccccagtgggagattaatgcggcaggatccagtggttcatttgtctccaaacaaacaagggcattctgatagccactactcgagccactccagtagcaatactctctccagcaatgcgtcaagtgcccatagtgatgagaagtggtacgatggggaccgcacagaatccgaactcaacagctataactatctgcaaggcacctctgctgacagtggcattgacaccacctcttatggccccagccacggcagcacagcctcgctgggggctgccacatcgtcacctcgctcagggccaggcaaggagaaagtggcacccctatggcacagctccagtgaagtaatctccatggcagatcggactttggagacagagagccacggcctggaccggaaaacagagtcttccctgagcttagacatacacagcaagagccaagccggctcgacccctctgacaagggagaacagcaccttcagtataaacgatgctgcttcccacacaagtaccatgagctcccgacactctgccagcccagtggttttcaccagtgcccggagttcacctaaagaagagcttcatccagctgccccctcacagctcgcaccatccttctcctcctcttcctcctcctcctctggtcctaggagtttttaccctcgccagggcgctactagcaagtacctgattggatggaaaaaacccgaaggaaccataaactccgtgggatttatggacacgagaaagcgtcatcagagcgatggcaatgaaatagcccacaccaggctgcgtgcctcaaccagagacctccgggcatctcctaagccaacctccaagtccaccattgaagaagatctaaagaaactaattgatcttgaaagcccaactcctgaatcacagaagagttttaagttccacgcactctcctctcctcagtctcctttccccagcacccccacctcacggcgggccttgcacagaacactgtcggacgagagcatttacaatagccagagggagcactttttcacctccagggcgtcacttctggaccaagccctgcccaacgacgtcctcttcagtagcacgtacccttctctccccaagtcgctcccgttgaggaggccttcttacaccttaggaatgaaatcgctgcatggagagttctcagcctcggacagctccctcactgacatccaggagacccgcaggcagcctatgcccgaccctggcctgatgcccctgcctgacactgctgcagacttggattggtccaacctggtagatgctgccaaagcctatgaggtccagagagcctcattttttgctgctagtgatgaaaaccatcgccccttgagtgctgcatccaacagtgatcagctggaggaccaggctctggcccagatgaagccttacagcagcagtaaagactcctctcccactctggcttctaaagtggaccagctggaaggtatgctgaagatgcttcgggaagatttgaagaaggaaaaagaagacaaagctcaccttcaggcggaggtgcagcacctgcgagaggacaacctgaggctacaggaggagtcccagaacgcctcggacaagctgaagaagttcacagaatgggtcttcaacaccatagacatgagctagggaaggctgaggaggacaggagaagggcccagacactccctccagtgagtgtcctgcagcccttattccctccatagaaagcatcctcagagcaccttccctggcttcctactctgccccctttcggggagtgcacaacacaatagttgcagatcaacaatcatcacctgccttttgtagaaaagaaaaacaaaaaaagtaaataaaaattttaaacagtaaaataaaagtttaactgctaaaaaaaaaaaaaaaaaaaaaaaaa

SEQ ID NO: 2 - protein, 1804 aa, Homo sapiens
MTSLKRSQTERPLATDRASVVGTDGTPKVHTDDFYMRRFRSQNGSLGSSVMAPVGPPRSEGSHHITSTPGVPKMGVRARIADWPPRKENIKESSRSSQEIETSSCLDSLSSKSSPVSQGSSVSLNSNDSAMLKSIQNTLKNKTRPSENMDSRFLMPEAYPSSPRKALRRIRQRSNSDITISELDVDSFDECISPTYKTGPSLHREYGSTSSIDKQGTSGESFFDLLKGYKDDKSDRGPTPTKLSDFLITGGGKGSGFSLDVIDGPISQRENLRLFKEREKPLKRRSKSETGDSSIFRKLRNAKGEELGKSSDLEDNRSEDSVRPWTCPKCFAHYDVQSILFDLNEAIMNRHNVIKRRNTTTGASAAAVASLVSGPLSHSASFSSPMGSTEDLNSKGSLSMDQGDDKSNELVMSCPYFRNEIGGEGERKISLSKSNSGSFSGCESASFESTLSSHCTNAGVAVLEVPKENLVLHLDRVKRYIVEHVDLGAYYYRKFFYQKEHWNYFGADENLGPVAVSIRREKPDEMKENGSPYNYRIIFRTSELMTLRGSVLEDAIPSTAKHSTARGLPLKEVLEHVVPELNVQCLRLAFNTPKVTEQLMKLDEQGLNYQQKVGIMYCKAGQSTEEEMYNNESAGPAFEEFLQLLGERVRLKGFEKYRAQLDTKTDSTGTHSLYTTYKDYEIMFHVSTMLPYTPNNKQQLLRKRHIGNDIVTIVFQEPGAQPFSPKNIRSHFQHVFVIVRVHNPCSDSVCYSVAVTRSRDVPSFGPPIPKGVTFPKSNVFRDFLLAKVINAENAAHKSEKFRAMATRTRQEYLKDLAEKNVTNTPIDPSGKFPFISLASKKKEKSKPYPGAELSSMGAIVWAVRAEDYNKAMELDCLLGISNEFIVLIEQETKSVVFNCSCRDVIGWTSTDTSLKIFYERGECVSVGSFINIEEIKEIVKRLQFVSKGCESVEMTLRRNGLGQLGFHVNYEGIVADVEPYGYAWQAGLRQGSRLVEICKVAVATLSHEQMIDLLRTSVTVKVVIIPPHDDCTPRRSCSETYRMPVMEYKMNEGVSYEFKFPFRNNNKWQRNASKGPHSPQVPSQVQSPMTSRLNAGKGDGKMPPPERAANIPRSISSDGRPLERRLSPGSDIYVTVSSMALARSQCRNSPSNLSSSSDTGSVGGTYRQKSMPEGFGVSRRSPASIDRQNTQSDIGGSGKSTPSWQRSEDSIADQMAYSYRGPQDFNSFVLEQHEYTEPTCHLPAVSKVLPAFRESPSGRLMRQDPVVHLSPNKQGHSDSHYSSHSSSNTLSSNASSAHSDEKWYDGDRTESELNSYNYLQGTSADSGIDTTSYGPSHGSTASLGAATSSPRSGPGKEKVAPLWHSSSEVISMADRTLETESHGLDRKTESSLSLDIHSKSQAGSTPLTRENSTFSINDAASHTSTMSSRHSASPVVFTSARSSPKEELHPAAPSQLAPSFSSSSSSSSGPRSFYPRQGATSKYLIGWKKPEGTINSVGFMDTRKRHQSDGNEIAHTRLRASTRDLRASPKPTSKSTIEEDLKKLIDLESPTPESQKSFKFHALSSPQSPFPSTPTSRRALHRTLSDESIYNSQREHFFTSRASLLDQALPNDVLFSSTYPSLPKSLPLRRPSYTLGMKSLHGEFSASDSSLTDIQETRRQPMPDPGLMPLPDTAADLDWSNLVDAAKAYEVQRASFFAASDENHRPLSAASNSDQLEDQALAQMKPYSSSKDSSPTLASKVDQLEGMLKMLREDLKKEKEDKAHLQAEVQHLREDNLRLQEESQNASDKLKKFTEWVFNTIDMS

SEQ ID NO: 3 - DNA, 5833 bp, Homo sapiens
  CDS (349)..(5697)
ggtgtggacgttgtctaaatttcggtagccatggcacaagaatataagaaagcatgggattatggcaaccacagaatctcagtagtacaagttccattcagttttttctgaaagaaagccctctgttaaagtgaagcaaagaaactgttgtggattataacgtttagaagttccaatttttcagtgctttacaaataaagcatcatttaaccttttaaatgaaaaagattaagatctcatgcaactgttgtattttctggaagccattctccaaaagggaagtgcacatttaaaacacagatatgatggtccttgctgcagggatttaagtctacttgcttttacatcatgaccagcttgaaacggtcacagacagaaaggcctcttgccactgacagggcctctgttgttggcacagacggcacccccaaagtccacactgatgatttctacatgcggcgcttccggtcccaaaatggcagcttaggatcatcagttatggctcctgtaggacccccccgaagtgaaggttctcaccatataacctcaacccccggagtcccaaaaatgggggtaagggcaaggattgcagattggcccccaagaaaggaaaacataaaagaatctagccgttcaagccaggaaatagaaacctcaagttgccttgatagcctgtcctccaaaagcagtcctgtgagtcagggaagttctgttagcctcaattccaatgactcagccatgctgaaaagcatacagaacacgctgaaaaacaagacaagaccgtcggagaacatggactccagatttctcatgcctgaagcctaccccagctcccccagaaaagctcttcgcagaatacgccagcgaagcaacagtgatatcaccataagtgaacttgatgtggatagctttgatgaatgtatctcacctacatacaagactggaccatcactgcacagggaatatggtagcacatcttcaattgataaacagggaacatctggagaaagcttttttgatttgttaaagggctacaaagatgacaaatctgatcgaggtccaactccaaccaagctcagtgactttctcattactggtggtggcaagggttctggtttctctttggatgtaatagacgggcctatctcacagagagagaacctcaggctttttaaggaaagggaaaaaccactcaagcgacgttcaaaatctgaaactggagactcctctatttttcgtaaattgcgcaatgccaaaggtgaagaacttgggaagtcatcagatcttgaagataaccgatcagaagactctgtcaggccctggacatgtccaaagtgctttgcccactatgatgtccagagtatattatttgatttgaatgaggcaattatgaacaggcacaatgttattaagaggagaaacaccaccactggagcttccgcagctgccgtggcatccttggtctctggacctctgtctcattcagccagttttagctccccaatgggcagcacagaggacctgaattccaaaggaagcctcagcatggaccagggagatgataaaagcaatgagcttgtaatgagctgtccatattttcggaatgagataggtggagaaggggagaggaaaatcagcctttcaaaatcaaattctggctcctttagtggatgtgaaagtgcctcctttgagtctacccttagttcccattgcacaaatgcaggagtggcagtacttgaagtgcccaaggagaacttggtgttgcacctagatagagtgaaaagatacatcgtggaacacgtagatctgggtgcatactattatagaaaatttttctaccagaaggaacactggaactattttggggctgatgagaatcttggtccagtggctgtgagcattcgaagggaaaaaccagatgaaatgaaagaaaatggatctccgtacaactaccgaataatttttagaactagtgagctcatgacactgagaggttcggtcctggaggacgccattccgtcgacagccaagcactcgacagccagaggcctgcctctcaaagaagtgctggagcacgtggttcctgagctcaatgtccagtgcctgcggttggccttcaacacacccaaggtcacagagcagctcatgaaactggatgaacaagggctgaactaccagcagaaagtaggcatcatgtactgcaaagctggacagagcactgaagaagagatgtacaacaatgagtcagctggcccagcctttgaagaattccttcaactattgggagagcgagttcggctcaaaggatttgagaagtatcgagcacagcttgataccaaaactgactccactggaacccattctctgtacacaacatacaaagattatgaaattatgttccatgtttctaccatgctgccatacacacccaacaacaaacaacagctcctgaggaagcggcacattggaaatgatatcgtaacaattgttttccaagagcctggagcacagccattcagcccaaaaaacatccgatcccacttccagcacgttttcgtcatcgtcagggtgcacaatccgtgctctgacagtgtctgttatagtgtggctgttaccaggtccagagatgtgccttcctttgggcctcccattcctaaaggggtcactttccctaagtcaaatgtgttcagggacttccttttggcgaaagtgattaatgcagaaaatgctgctcataaatcggagaagtttcgggccatggcaactcggacccgccaggaatacctgaaagatctggcagaaaagaatgtcaccaacacccctatcgacccttctggcaagtttccgttcatctctctggcttccaagaagaaggaaaagtctaagccatatccaggagccgagctcagcagcatgggggccattgtatgggcagtccgggctgaagactacaacaaggccatggaactagactgccttttagggatctccaatgagttcattgtgctcattgaacaggaaacaaagagcgtggtcttcaattgttcctgtagagatgtgatagggtggacttcaactgacaccagcctcaaaatcttctatgaacgaggagaatgtgtttcagtgggtagttttattaacattgaggagatcaaagagattgtcaaaaggttgcagtttgtttcaaaaggctgtgaatcggtggagatgactctgcgaagaaatgggctaggacagcttggcttccatgtcaactatgagggcattgtggcggatgtggagccctacggttatgcctggcaggcagggctgaggcagggcagtcgcctggtggagatctgcaaggtggcggtagccactctgagccatgagcagatgatcgacctcctgagaacatctgtcacggtgaaggttgtcatcattcccccgcatgatgactgcaccccgcggaggagttgctctgaaacctaccgcatgccagtgatggagtacaaaatgaatgaaggtgtttcatacgaattcaagtttcccttccgaaataataacaagtggcagaggaacgccagcaaggggcctcattcacctcaagtcccgtcccaggtgcagagtcccatgacctcgcggctgaatgctggaaaaggagatgggaagatgcctcctccagaaagagccgccaacatccctcgaagcatctccagtgacgggcgcccactagagaggcggctgtctcctggttcggacatctatgtgacggtctcatccatggctttagcaagatcccagtgtcggaactctcctagcaacttgtcttcatccagtgatactggttctgtggggggcacttacaggcagaagtccatgcccgaagggtttggagtgagccgtagatccccagcctccattgacaggcagaacacccagtcagatattggtggcagcggaaaatccacgcctagctggcaaagaagtgaggatagcattgctgaccagatggagccaacatgccatctcccagcagtatcaaaggtactgccagctttccgagagagccccagtgggagattaatgcggcaggatccagtggttcatttgtctccaaacaaacaagggcattctgatagccactactcgagccactccagtagcaatactctctccagcaatgcgtcaagtgcccatagtgatgagaagtggtacgatggggaccgcacagaatccgaactcaacagctataactatctgcaaggcacctctgctgacagtggcattgacaccacctcttatggccccagccacggcagcacagcctcgctgggggctgccacatcgtcacctcgctcagggccaggcaaggagaaagtggcacccctatggcacagctccagtgaagtaatctccatggcagatcggactttggagacagagagccacggcctggaccggaaaacagagtcttccctgagcttagacatacacagcaagagccaagccggctcgacccctctgacaagggagaacagcaccttcagtataaacgatgctgcttcccacacaagtaccatgagctcccgacactctgccagcccagtggttttcaccagtgcccggagttcacctaaagaagagcttcatccagctgccccctcacagctcgcaccatccttctcctcctcttcctcctcctcctctggtcctaggagtttttaccctcgccagggcgctactagcaagtacctgattggatggaaaaaacccgaaggaaccataaactccgtgggatttatggacacgagaaagcgtcatcagagcgatggcaatgaaatagcccacaccaggctgcgtgcctcaaccagagacctccgggcatctcctaagccaacctccaagtccaccattgaagaagatctaaagaaactaattgatcttgaaagcccaactcctgaatcacagaagagttttaagttccacgcactctcctctcctcagtctcctttccccagcacccccacctcacggcgggccttgcacagaacactgtcggacgagagcatttacaatagccagagggagcactttttcacctccagggcgtcacttctggaccaagccctgcccaacgacgtcctcttcagtagcacgtacccttctctccccaagtcgctcccgttgaggaggccttcttacaccttaggaatgaaatcgctgcatggagagttctcagcctcggacagctccctcactgacatccaggagacccgcaggcagcctatgcccgaccctggcctgatgcccctgcctgacactgctgcagacttggattggtccaacctggtagatgctgccaaagcctatgaggtccagagagcctcattttttgctgctagtgatgaaaaccatcgccccttgagtgctgcatccaacagtgatcagctggaggaccaggctctggcccagatgaagccttacagcagcagtaaagactcctctcccactctggcttctaaagtggaccagctggaaggtatgctgaagatgcttcgggaagatttgaagaaggaaaaagaagacaaagctcaccttcaggcggaggtgcagcacctgcgagaggacaacctgaggctacaggaggagtcccagaacgcctcggacaagctgaagaagttcacagaatgggtcttcaacaccatagacatgagctagggaaggctgaggaggacaggagaagggcccagacactccctccagtgagtgtcctgcagcccttattccctccatagaaagcatcctcagagcaccttccctggcttcctactctgccccctttcggggagtg

SEQ ID NO: 4 - protein, 1783 aa, Homo sapiens
MTSLKRSQTERPLATDRASVVGTDGTPKVHTDDFYMRRFRSQNGSLGSSVMAPVGPPRSEGSHHITSTPGVPKMGVRARIADWPPRKENIKESSRSSQEIETSSCLDSLSSKSSPVSQGSSVSLNSNDSAMLKSIQNTLKNKTRPSENMDSRFLMPEAYPSSPRKALRRIRQRSNSDITISELDVDSFDECISPTYKTGPSLHREYGSTSSIDKQGTSGESFFDLLKGYKDDKSDRGPTPTKLSDFLITGGGKGSGFSLDVIDGPISQRENLRLFKEREKPLKRRSKSETGDSSIFRKLRNAKGEELGKSSDLEDNRSEDSVRPWTCPKCFAHYDVQSILFDLNEAIMNRHNVIKRRNTTTGASAAAVASLVSGPLSHSASFSSPMGSTEDLNSKGSLSMDQGDDKSNELVMSCPYFRNEIGGEGERKISLSKSNSGSFSGCESASFESTLSSHCTNAGVAVLEVPKENLVLHLDRVKRYIVEHVDLGAYYYRKFFYQKEHWNYFGADENLGPVAVSIRREKPDEMKENGSPYNYRIIFRTSELMTLRGSVLEDAIPSTAKHSTARGLPLKEVLEHVVPELNVQCLRLAFNTPKVTEQLMKLDEQGLNYQQKVGIMYCKAGQSTEEEMYNNESAGPAFEEFLQLLGERVRLKGFEKYRAQLDTKTDSTGTHSLYTTYKDYEIMFHVSTMLPYTPNNKQQLLRKRHIGNDIVTIVFQEPGAQPFSPKNIRSHFQHVFVIVRVHNPCSDSVCYSVAVTRSRDVPSFGPPIPKGVTFPKSNVFRDFLLAKVINAENAAHKSEKFRAMATRTRQEYLKDLAEKNVTNTPIDPSGKFPFISLASKKKEKSKPYPGAELSSMGAIVWAVRAEDYNKAMELDCLLGISNEFIVLIEQETKSVVFNCSCRDVIGWTSTDTSLKIFYERGECVSVGSFINIEEIKEIVKRLQFVSKGCESVEMTLRRNGLGQLGFHVNYEGIVADVEPYGYAWQAGLRQGSRLVEICKVAVATLSHEQMIDLLRTSVTVKVVIIPPHDDCTPRRSCSETYRMPVMEYKMNEGVSYEFKFPFRNNNKWQRNASKGPHSPQVPSQVQSPMTSRLNAGKGDGKMPPPERAANIPRSISSDGRPLERRLSPGSDIYVTVSSMALARSQCRNSPSNLSSSSDTGSVGGTYRQKSMPEGFGVSRRSPASIDRQNTQSDIGGSGKSTPSWQRSEDSIADQMEPTCHLPAVSKVLPAFRESPSGRLMRQDPVVHLSPNKQGHSDSHYSSHSSSNTLSSNASSAHSDEKWYDGDRTESELNSYNYLQGTSADSGIDTTSYGPSHGSTASLGAATSSPRSGPGKEKVAPLWHSSSEVISMADRTLETESHGLDRKTESSLSLDIHSKSQAGSTPLTRENSTFSINDAASHTSTMSSRHSASPVVFTSARSSPKEELHPAAPSQLAPSFSSSSSSSSGPRSFYPRQGATSKYLIGWKKPEGTINSVGFMDTRKRHQSDGNEIAHTRLRASTRDLRASPKPTSKSTIEEDLKKLIDLESPTPESQKSFKFHALSSPQSPFPSTPTSRRALHRTLSDESIYNSQREHFFTSRASLLDQALPNDVLFSSTYPSLPKSLPLRRPSYTLGMKSLHGEFSASDSSLTDIQETRRQPMPDPGLMPLPDTAADLDWSNLVDAAKAYEVQRASFFAASDENHRPLSAASNSDQLEDQALAQMKPYSSSKDSSPTLASKVDQLEGMLKMLREDLKKEKEDKAHLQAEVQHLREDNLRLQEESQNASDKLKKFTEWVFNTIDMS

SEQ ID NO: 5 - DNA, 5412 bp, Homo sapiens
  CDS (1)..(5412)
atgaccagcttgaaacggtcacagacagaaaggcctcttgccactgacagggcctctgttgttggcacagacggcacccccaaagtccacactgatgatttctacatgcggcgcttccggtcccaaaatggcagcttaggatcatcagttatggctcctgtaggacccccccgaagtgaaggttctcaccatataacctcaacccccggagtcccaaaaatgggggtaagggcaaggattgcagattggcccccaagaaaggaaaacataaaagaatctagccgttcaagccaggaaatagaaacctcaagttgccttgatagcctgtcctccaaaagcagtcctgtgagtcagggaagttctgttagcctcaattccaatgactcagccatgctgaaaagcatacagaacacgctgaaaaacaagacaagaccgtcggagaacatggactccagatttctcatgcctgaagcctaccccagctcccccagaaaagctcttcgcagaatacgccagcgaagcaacagtgatatcaccataagtgaacttgatgtggatagctttgatgaatgtatctcacctacatacaagactggaccatcactgcacagggaatatggtagcacatcttcaattgataaacagggaacatctggagaaagcttttttgatttgttaaagggctacaaagatgacaaatctgatcgaggtccaactccaaccaagctcagtgactttctcattactggtggtggcaagggttctggtttctctttggatgtaatagacgggcctatctcacagagagagaacctcaggctttttaaggaaagggaaaaaccactcaagcgacgttcaaaatctgaaactggagactcctctatttttcgtaaattgcgcaatgccaaaggtgaagaacttgggaagtcatcagatcttgaagataaccgatcagaagactctgtcaggccctggacatgtccaaagtgctttgcccactatgatgtccagagtatattatttgatttgaatgaggcaattatgaacaggcacaatgttattaagaggagaaacaccaccactggagcttccgcagctgccgtggcatccttggtctctggacctctgtctcattcagccagttttagctccccaatgggcagcacagaggacctgaattccaaaggaagcctcagcatggaccagggagatgataaaagcaatgagcttgtaatgagctgtccatattttcggaatgagataggtggagaaggggagaggaaaatcagcctttcaaaatcaaattctggctcctttagtggatgtgaaagtgcctcctttgagtctacccttagttcccattgcacaaatgcaggagtggcagtacttgaagtgcccaaggagaacttggtgttgcacctagatagagtgaaaagatacatcgtggaacacgtagatctgggtgcatactattatagaaaatttttctaccagaaggaacactggaactattttggggctgatgagaatcttggtccagtggctgtgagcattcgaagggaaaaaccagatgaaatgaaagaaaatggatctccgtacaactaccgaataatttttagaactagtgagctcatgacactgagaggttcggtcctggaggacgccattccgtcgacagccaagcactcgacagccagaggcctgcctctcaaagaagtgctggagcacgtggttcctgagctcaatgtccagtgcctgcggttggccttcaacacacccaaggtcacagagcagctcatgaaactggatgaacaagggctgaactaccagcagaaagtaggcatcatgtactgcaaagctggacagagcactgaagaagagatgtacaacaatgagtcagctggcccagcctttgaagaattccttcaactattgggagagcgagttcggctcaaaggatttgagaagtatcgagcacagcttgataccaaaactgactccactggaacccattctctgtacacaacatacaaagattatgaaattatgttccatgtttctaccatgctgccatacacacccaacaacaaacaacagctcctgaggaagcggcacattggaaatgatatcgtaacaattgttttccaagagcctggagcacagccattcagcccaaaaaacatccgatcccacttccagcacgttttcgtcatcgtcagggtgcacaatccgtgctctgacagtgtctgttatagtgtggctgttaccaggtccagagatgtgccttcctttgggcctcccattcctaaaggggtcactttccctaagtcaaatgtgttcagggacttccttttggcgaaagtgattaatgcagaaaatgctgctcataaatcggagaagtttcgggccatggcaactcggacccgccaggaatacctgaaagatctggcagaaaagaatgtcaccaacacccctatcgacccttctggcaagtttccgttcatctctctggcttccaagaagaaggaaaagtctaagccatatccaggagccgagctcagcagcatgggggccattgtatgggcagtccgggctgaagactacaacaaggccatggaactagactgccttttagggatctccaatgagttcattgtgctcattgaacaggaaacaaagagcgtggtcttcaattgttcctgtagagatgtgatagggtggacttcaactgacaccagcctcaaaatcttctatgaacgaggagaatgtgtttcagtgggtagttttattaacattgaggagatcaaagagattgtcaaaaggttgcagtttgtttcaaaaggctgtgaatcggtggagatgactctgcgaagaaatgggctaggacagcttggcttccatgtcaactatgagggcattgtggcggatgtggagccctacggttatgcctggcaggcagggctgaggcagggcagtcgcctggtggagatctgcaaggtggcggtagccactctgagccatgagcagatgatcgacctcctgagaacatctgtcacggtgaaggttgtcatcattcccccgcatgatgactgcaccccgcggaggagttgctctgaaacctaccgcatgccagtgatggagtacaaaatgaatgaaggtgtttcatacgaattcaagtttcccttccgaaataataacaagtggcagaggaacgccagcaaggggcctcattcacctcaagtcccgtcccaggtgcagagtcccatgacctcgcggctgaatgctggaaaaggagatgggaagatgcctcctccagaaagagccgccaacatccctcgaagcatctccagtgacgggcgcccactagagaggcggctgtctcctggttcggacatctatgtgacggtctcatccatggctttagcaagatcccagtgtcggaactctcctagcaacttgtcttcatccagtgatactggttctgtggggggcacttacaggcagaagtccatgcccgaagggtttggagtgagccgtagatccccagcctccattgacaggcagaacacccagtcagatattggtggcagcggaaaatccacgcctagctggcaaagaagtgaggatagcattgctgaccagatggcttacagttatagaggacctcaggatttcaattcttttgtcctcgagcagcatgaatatacagagccaacatgccatctcccagcagtatcaaaggtactgccagctttccgagagagccccagtgggagattaatgcggcaggatccagtggttcatttgtctccaaacaaacaagggcattctgatagccactactcgagccactccagtagcaatactctctccagcaatgcgtcaagtgcccatagtgatgagaagtggtacgatggggaccgcacagaatccgaactcaacagctataactatctgcaaggcacctctgctgacagtggcattgacaccacctcttatggccccagccacggcagcacagcctcgctgggggctgccacatcgtcacctcgctcagggccaggcaaggagaaagtggcacccctatggcacagctccagtgaagtaatctccatggcagatcggactttggagacagagagccacggcctggaccggaaaacagagtcttccctgagcttagacatacacagcaagagccaagccggctcgacccctctgacaagggagaacagcaccttcagtataaacgatgctgcttcccacacaagtaccatgagctcccgacactctgccagcccagtggttttcaccagtgcccggagttcacctaaagaagagcttcatccagctgccccctcacagctcgcaccatccttctcctcctcttcctcctcctcctctggtcctaggagtttttaccctcgccagggcgctactagcaagtacctgattggatggaaaaaacccgaaggaaccataaactccgtgggatttatggacacgagaaagcgtcatcagagcgatggcaatgaaatagcccacaccaggctgcgtgcctcaaccagagacctccgggcatctcctaagccaacctccaagtccaccattgaagaagatctaaagaaactaattgatcttgaaagcccaactcctgaatcacagaagagttttaagttccacgcactctcctctcctcagtctcctttccccagcacccccacctcacggcgggccttgcacagaacactgtcggacgagagcatttacaatagccagagggagcactttttcacctccagggcgtcacttctggaccaagccctgcccaacgacgtcctcttcagtagcacgtacccttctctccccaagtcgctcccgttgaggaggccttcttacaccttaggaatgaaatcgctgcatggagagttctcagcctcggacagctccctcactgacatccaggagacccgcaggcagcctatgcccgaccctggcctgatgcccctgcctgacactgctgcagacttggattggtccaacctggtagatgctgccaaagcctatgaggtccagagagcctcattttttgctgctagtgatgaaaaccatcgccccttgagtgctgcatccaacagtgatcagctggaggaccaggctctggcccagatgaagccttacagcagcagtaaagactcctctcccactctggcttctaaagtggaccagctggaaggtatgctgaagatgcttcgggaagatttgaagaaggaaaaagaagacaaagctcaccttcaggcggaggtgcagcacctgcgagaggacaacctgaggctacaggaggagtcccagaacgcctcggacaagctgaagaagttcacagaatgggtcttcaacaccatagacatgagc

SEQ ID NO: 6 - DNA, 5349 bp, Homo sapiens
  CDS (1)..(5349)
atgaccagcttgaaacggtcacagacagaaaggcctcttgccactgacagggcctctgttgttggcacagacggcacccccaaagtccacactgatgatttctacatgcggcgcttccggtcccaaaatggcagcttaggatcatcagttatggctcctgtaggacccccccgaagtgaaggttctcaccatataacctcaacccccggagtcccaaaaatgggggtaagggcaaggattgcagattggcccccaagaaaggaaaacataaaagaatctagccgttcaagccaggaaatagaaacctcaagttgccttgatagcctgtcctccaaaagcagtcctgtgagtcagggaagttctgttagcctcaattccaatgactcagccatgctgaaaagcatacagaacacgctgaaaaacaagacaagaccgtcggagaacatggactccagatttctcatgcctgaagcctaccccagctcccccagaaaagctcttcgcagaatacgccagcgaagcaacagtgatatcaccataagtgaacttgatgtggatagctttgatgaatgtatctcacctacatacaagactggaccatcactgcacagggaatatggtagcacatcttcaattgataaacagggaacatctggagaaagcttttttgatttgttaaagggctacaaagatgacaaatctgatcgaggtccaactccaaccaagctcagtgactttctcattactggtggtggcaagggttctggtttctctttggatgtaatagacgggcctatctcacagagagagaacctcaggctttttaaggaaagggaaaaaccactcaagcgacgttcaaaatctgaaactggagactcctctatttttcgtaaattgcgcaatgccaaaggtgaagaacttgggaagtcatcagatcttgaagataaccgatcagaagactctgtcaggccctggacatgtccaaagtgctttgcccactatgatgtccagagtatattatttgatttgaatgaggcaattatgaacaggcacaatgttattaagaggagaaacaccaccactggagcttccgcagctgccgtggcatccttggtctctggacctctgtctcattcagccagttttagctccccaatgggcagcacagaggacctgaattccaaaggaagcctcagcatggaccagggagatgataaaagcaatgagcttgtaatgagctgtccatattttcggaatgagataggtggagaaggggagaggaaaatcagcctttcaaaatcaaattctggctcctttagtggatgtgaaagtgcctcctttgagtctacccttagttcccattgcacaaatgcaggagtggcagtacttgaagtgcccaaggagaacttggtgttgcacctagatagagtgaaaagatacatcgtggaacacgtagatctgggtgcatactattatagaaaatttttctaccagaaggaacactggaactattttggggctgatgagaatcttggtccagtggctgtgagcattcgaagggaaaaaccagatgaaatgaaagaaaatggatctccgtacaactaccgaataatttttagaactagtgagctcatgacactgagaggttcggtcctggaggacgccattccgtcgacagccaagcactcgacagccagaggcctgcctctcaaagaagtgctggagcacgtggttcctgagctcaatgtccagtgcctgcggttggccttcaacacacccaaggtcacagagcagctcatgaaactggatgaacaagggctgaactaccagcagaaagtaggcatcatgtactgcaaagctggacagagcactgaagaagagatgtacaacaatgagtcagctggcccagcctttgaagaattccttcaactattgggagagcgagttcggctcaaaggatttgagaagtatcgagcacagcttgataccaaaactgactccactggaacccattctctgtacacaacatacaaagattatgaaattatgttccatgtttctaccatgctgccatacacacccaacaacaaacaacagctcctgaggaagcggcacattggaaatgatatcgtaacaattgttttccaagagcctggagcacagccattcagcccaaaaaacatccgatcccacttccagcacgttttcgtcatcgtcagggtgcacaatccgtgctctgacagtgtctgttatagtgtggctgttaccaggtccagagatgtgccttcctttgggcctcccattcctaaaggggtcactttccctaagtcaaatgtgttcagggacttccttttggcgaaagtgattaatgcagaaaatgctgctcataaatcggagaagtttcgggccatggcaactcggacccgccaggaatacctgaaagatctggcagaaaagaatgtcaccaacacccctatcgacccttctggcaagtttccgttcatctctctggcttccaagaagaaggaaaagtctaagccatatccaggagccgagctcagcagcatgggggccattgtatgggcagtccgggctgaagactacaacaaggccatggaactagactgccttttagggatctccaatgagttcattgtgctcattgaacaggaaacaaagagcgtggtcttcaattgttcctgtagagatgtgatagggtggacttcaactgacaccagcctcaaaatcttctatgaacgaggagaatgtgtttcagtgggtagttttattaacattgaggagatcaaagagattgtcaaaaggttgcagtttgtttcaaaaggctgtgaatcggtggagatgactctgcgaagaaatgggctaggacagcttggcttccatgtcaactatgagggcattgtggcggatgtggagccctacggttatgcctggcaggcagggctgaggcagggcagtcgcctggtggagatctgcaaggtggcggtagccactctgagccatgagcagatgatcgacctcctgagaacatctgtcacggtgaaggttgtcatcattcccccgcatgatgactgcaccccgcggaggagttgctctgaaacctaccgcatgccagtgatggagtacaaaatgaatgaaggtgtttcatacgaattcaagtttcccttccgaaataataacaagtggcagaggaacgccagcaaggggcctcattcacctcaagtcccgtcccaggtgcagagtcccatgacctcgcggctgaatgctggaaaaggagatgggaagatgcctcctccagaaagagccgccaacatccctcgaagcatctccagtgacgggcgcccactagagaggcggctgtctcctggttcggacatctatgtgacggtctcatccatggctttagcaagatcccagtgtcggaactctcctagcaacttgtcttcatccagtgatactggttctgtggggggcacttacaggcagaagtccatgcccgaagggtttggagtgagccgtagatccccagcctccattgacaggcagaacacccagtcagatattggtggcagcggaaaatccacgcctagctggcaaagaagtgaggatagcattgctgaccagatggagccaacatgccatctcccagcagtatcaaaggtactgccagctttccgagagagccccagtgggagattaatgcggcaggatccagtggttcatttgtctccaaacaaacaagggcattctgatagccactactcgagccactccagtagcaatactctctccagcaatgcgtcaagtgcccatagtgatgagaagtggtacgatggggaccgcacagaatccgaactcaacagctataactatctgcaaggcacctctgctgacagtggcattgacaccacctcttatggccccagccacggcagcacagcctcgctgggggctgccacatcgtcacctcgctcagggccaggcaaggagaaagtggcacccctatggcacagctccagtgaagtaatctccatggcagatcggactttggagacagagagccacggcctggaccggaaaacagagtcttccctgagcttagacatacacagcaagagccaagccggctcgacccctctgacaagggagaacagcaccttcagtataaacgatgctgcttcccacacaagtaccatgagctcccgacactctgccagcccagtggttttcaccagtgcccggagttcacctaaagaagagcttcatccagctgccccctcacagctcgcaccatccttctcctcctcttcctcctcctcctctggtcctaggagtttttaccctcgccagggcgctactagcaagtacctgattggatggaaaaaacccgaaggaaccataaactccgtgggatttatggacacgagaaagcgtcatcagagcgatggcaatgaaatagcccacaccaggctgcgtgcctcaaccagagacctccgggcatctcctaagccaacctccaagtccaccattgaagaagatctaaagaaactaattgatcttgaaagcccaactcctgaatcacagaagagttttaagttccacgcactctcctctcctcagtctcctttccccagcacccccacctcacggcgggccttgcacagaacactgtcggacgagagcatttacaatagccagagggagcactttttcacctccagggcgtcacttctggaccaagccctgcccaacgacgtcctcttcagtagcacgtacccttctctccccaagtcgctcccgttgaggaggccttcttacaccttaggaatgaaatcgctgcatggagagttctcagcctcggacagctccctcactgacatccaggagacccgcaggcagcctatgcccgaccctggcctgatgcccctgcctgacactgctgcagacttggattggtccaacctggtagatgctgccaaagcctatgaggtccagagagcctcattttttgctgctagtgatgaaaaccatcgccccttgagtgctgcatccaacagtgatcagctggaggaccaggctctggcccagatgaagccttacagcagcagtaaagactcctctcccactctggcttctaaagtggaccagctggaaggtatgctgaagatgcttcgggaagatttgaagaaggaaaaagaagacaaagctcaccttcaggcggaggtgcagcacctgcgagaggacaacctgaggctacaggaggagtcccagaacgcctcggacaagctgaagaagttcacagaatgggtcttcaacaccatagacatgagc

SEQ ID NO: 7 - DNA, 14 bp, Homo sapiens
gacggaacagcttc

SEQ ID NO: 8 - DNA, 13 bp, Homo sapiens
tgcgtctggttct